(19) United States
(12) Patent Application Publication
(10) Pub. No.: US 2016/0328523 A1
(43) Pub. Date: Nov. 10, 2016

(54) SYSTEM AND METHOD FOR DOCUMENTING PATIENT INFORMATION

(71) Applicant: VYNCA, LLC, Mountain View, CA (US)

(72) Inventors: Rush L. Bartlett, II, Mountain View, CA (US); David Lin, Cupertino, CA (US); Jack Yeh, Mountain View, CA (US); Ryan J.F. Van Wert, Palo Alto, CA (US); Frank T. Wang, Cupertino, CA (US)

(21) Appl. No.: 15/204,960

(22) Filed: Jul. 7, 2016

Related U.S. Application Data
(63) Continuation of application No. 14/685,405, filed on Apr. 13, 2015.
(60) Provisional application No. 62/069,518, filed on Oct. 28, 2014.
(60) Provisional application No. 61/978,961, filed on Apr. 13, 2014.

(51) Int. Cl. G06F 19/00; H04L 29/08; H04L 29/06; G06F 17/30

(52) U.S. Cl. CPC G06F 19/322; G06F 17/30887; H04L 67/02; G06F 17/30867; H04L 63/0471; H04L 63/061; H04L 63/0442

(57) ABSTRACT

A method for providing access to patient information from within an electronic medical record may involve: receiving, from a user, at least one piece of identifying information, identifying the user as a person authorized to access the patient information; providing an encrypted link on an electronic medical record of the patient, wherein the encrypted link is preloaded with the at least one piece of identifying information and a patient medical record number corresponding to the patient; decrypting the encrypted link in response to the user clicking on the encrypted link, without requiring the user to provide any further identifying information; and providing the patient information to the user via a secure web site, in response to the user clicking on the link.

### CROSS-REFERENCE TO RELATED APPLICATION(S)

This application is a continuation of U.S. patent application Ser. No. 14/685,405, filed Apr. 13, 2015, and entitled “SYSTEM AND METHOD FOR DOCUMENTING PATIENT INFORMATION,” which claims priority to U.S. Provisional Patent Application No. 61/978,961, entitled “System and Method for Documenting Patient Information,” filed on Apr. 13, 2014, and U.S. Provisional Patent Application No. 62/069,518, entitled “System and Method for Documenting Patient Information,” filed Oct. 28, 2014. The full disclosures of the above-listed patent applications are hereby incorporated by reference herein.

### BACKGROUND

Exchange of information across different computer applications and web sites can often be challenging. For example, one application may require information, such as demographic data, from another application, but the two applications may be incompatible with each other. Oftentimes, the information exchanged across applications includes private, confidential or otherwise sensitive information, for example in the healthcare industry, in governmental agencies, or even the extremely common transfer of credit card information. Currently available information exchange platforms do a poor job of allowing for secure exchange of information between different, often incompatible applications. Integration of such applications may be difficult or impossible, for example, due to proprietary software, incompatibility of programming languages or platforms, and/or limited information technology resources.

Therefore, there is a need to develop systems and methods for information exchange between software applications that does not require specific modification or manipulation of existing applications, web sites or servers themselves. Such a need exists in the healthcare, financial, and retail industries, government, and many other areas. In this regard, it would be advantageous to have a user interface that allowed for input and access of information by two or more different users using different applications. Ideally, such a user interface would be easy to use and would be compatible with many different applications. At least some of these objectives will be met by the embodiments described below.

### BRIEF SUMMARY

The disclosed systems and methods provide an improved user interface for information exchange across computer-based applications, web pages, web services or web servers. Generally, the systems and methods are directed to a unique user interface that facilitates efficient creation and dissemination of forms from a central source in a way that protects privacy and security and is compliant with relevant regulations, such as the federal Health Insurance Portability and Accountability Act (HIPAA).

Certain embodiments disclosed herein allow for document completion and form creation and access with enhanced features and media, while also offering information exchange across applications, web sites, and/or networks, by capturing relevant information on one or more user screens and delivering it to an application for storage and later access.

In one aspect, a method for providing access to patient information from within an electronic medical record may include receiving from a user at least one piece of identifying information, identifying the user as a person authorized to access the patient information; providing an encrypted link on an electronic medical record of the patient; decrypting the encrypted link in response to the user clicking on the encrypted link, without requiring the user to provide any further identifying information; and providing the patient information to the user via a secure web site, in response to the user clicking on the link. The encrypted link may be preloaded with the at least one piece of identifying information and a patient medical record number corresponding to the patient.

In another aspect, a method for providing access to patient information from within an electronic medical record to multiple users may include displaying a first link on an electronic medical record of a patient on a display monitor of a computer. The first link may connect a first user to a secure web site in response to clicking on the first link. The method may further include providing the patient information to the first user via the secure web site; and displaying a second link on the secure web site on the computer. The second link may link the computer with a mobile electronic device in response to the first user clicking on the second link. The method may further include displaying a display code on the mobile electronic device in response to the first user clicking on the second link; receiving an entered code, entered into the mobile electronic device by a second user; verifying that the entered code matches the display code; and linking the computer with the mobile electronic device, based on the verifying step.

### BRIEF DESCRIPTION OF THE DRAWINGS

FIG. 1A depicts a screen shot of a healthcare provider sign-in page of an electronic medical records management system, according to one embodiment;

FIG. 1B depicts a screen shot of a patient form retrieval page of an electronic medical records management system, according to one embodiment;

FIG. 2A depicts a screen shot of an activity dashboard page for Physician Orders for Life Sustaining Treatment (POLST) of an exemplary user interface and system, according to one embodiment;

FIG. 2B depicts a screen shot of the activity dashboard of FIG. 2A, but illustrating a yes/no feature used to identify an advance directive or a POLST form or other Advance Care Planning Document in the system;

FIG. 2C depicts a screen shot of an activity summary page for POLST, according to one embodiment;

FIG. 3 depicts a screen shot of an electronic medical records management system, according to one embodiment;

FIG. 4 depicts a screen shot of a medical interventions page of an electronic medical records management system, according to one embodiment;

FIG. 5 depicts a screen shot of an information confirmation page an electronic document management system, according to one embodiment;

FIG. 6 depicts a screen shot of an exemplary user interface and system for accessing an active medical form from a link within an electronic medical record, according to one embodiment;

FIG. 7 depicts a screen shot of an exemplary user interface and system for accessing active medical information, such as a POLST form, advanced directive, or health care proxy form, and any audit history or form creation or invalidation history, according to one embodiment;

FIG. 8 depicts a screen shot of an exemplary user interface and system for accessing active medical information, such as a healthcare proxy form, including an access point to create a new form, view old forms, view any additional information, or view an audit log of accesses, views, changes, and/or edits, according to one embodiment;

FIG. 9 depicts a screen shot of an exemplary user interface and system for accessing an active medical form, such as a POLST form, from a link or access point within an electronic medical record, including a visual cue, prior to the clicking of the link, as to the presence or absence of a document to be accessed from the link, where in the example shown, no link is presenting a visual clue as to the presence of a form indicating its absence, according to one embodiment;

FIG. 10 depicts a user interface and system for entering in document completion information, such as for a POLST or other medical form, including a patient's wishes for desired treatment to be entered by a physician, according to one embodiment;

FIG. 11 depicts an exemplary user interface and system for entering in document completion information such as for a POLST or other medical form while also demonstrating an active audit tracking capability to provide report information as to the quality of a conversation taking place when the form is being completed, according to one embodiment;

FIG. 12 depicts an exemplary user interface and system for entering patient information into a medical form tool where medical information can be selected by a physician or patient to demonstrate a patient's choices for care, according to one embodiment;

FIG. 13 depicts an exemplary user interface and system for documenting sensitive information, including a medical form, such that the legal language and/or terminology definitions are displayed within a link about the current active page or session, according to one embodiment;

FIG. 14 depicts an exemplary user interface and system for entering in information for a document electronically while also providing targeted just in time education for critical sections of the form and/or choices required including but not limited to medical interventions and videos or education around those choices as they pertain to advanced care planning documentation such as a POLST form, according to one embodiment;

FIGS. 15A-15C depict an exemplary user interface and system for documenting information, such as a POLST form, in an electronic form, such that integration with a system such as an electronic medical record allows for active pulling of patient and/or provider information into the record and auto-completing of that information within the form for review by the completing party, according to one embodiment;

FIG. 16 depicts an exemplary user interface and system for documenting medical information, including information useful for patient identification in an emergency, such as a picture, by linking a smartphone to the web session through secure QR code or other link, according to one embodiment;

FIG. 17 depicts an exemplary user interface and system on a smartphone for documenting medical information including a picture by linking the smartphone to the web session through secure QR code or other link and then taking a photo of a patient, which is automatically saved to the web session running both on the phone and/or another system, such that the picture image is not saved directly onto the hard drive of the smartphone itself, according to one embodiment;

FIG. 18 depicts an exemplary user interface and system for documenting medical information, including information useful for patient identification in an emergency, such as a picture, by linking a smartphone to the web session through secure QR code or other link and the display of that information, according to one embodiment;

FIG. 19 depicts an exemplary user interface and system for documenting medical information through a secure access portal including providing for a session log out and settings management tool for the securely authenticated user, according to one embodiment;

FIG. 20 depicts an exemplary user interface and system for documenting sensitive information such as a patient's choices on a POLST form including cardiopulmonary resuscitation while also providing detailed legal information on such a choice, according to one embodiment;

FIG. 21 depicts an exemplary user interface and system for documenting sensitive information such as a patient's choices on a POLST form including cardiopulmonary resuscitation while also providing just in time educational information such as videos on such a choice, according to one embodiment;

FIG. 22 depicts an exemplary user interface and system for documenting sensitive medical information such as a patient's choices on a POLST form and a translation feature that allows for multiple languages to be displayed simultaneously while completing these choices, according to one embodiment;

FIG. 23 depicts an exemplary user interface and system for documenting sensitive medical information such as a patient's choices on a POLST form and a security and error checking feature that forces compliance with accepted legal guidance around mutually exclusive choices such as requiring somebody to attempt CPR also defaults to full treatment in a secondary section in some states documentation, according to one embodiment;

FIG. 24 depicts an exemplary user interface and system for documenting sensitive medical information such as a patient's choices on a POLST form and a feature to customize the just in time medical education by various sites, according to one embodiment;

FIG. 25 depicts an exemplary user interface and system for documenting sensitive medical information such as a patient's choices on a POLST form for artificial nutrition including but not limited to entry of specific lengths of time desired while also providing just in time education and/or legal information on the effects of such a choice, according to one embodiment;

FIG. 26 depicts an exemplary user interface and system for entering demographic information to accompany the process of authenticating and/or signing a document digitally with a signature pad, touch screen, linked smartphone or tablet, and/or electronic documentation means, according to one embodiment;

FIG. 27 depicts an exemplary user interface and system for entering demographic information to accompany the process of authenticating and/or signing a document digitally including auto-populating and/or collapsing entry boxes if, within the sequence of choices, demographic information already entered or auto-populated within another section of the form can be auto-filled or completed into this section, according to one embodiment;

FIG. 28 depicts an exemplary user interface and system for electronically signing an authenticated document, according to one embodiment;

FIG. 29 depicts an exemplary user interface and system for electronically signing an authenticated document by linking a mobile device such as a smartphone or tablet via QR code to a web session to allow for data input from that mobile device to facilitate the unique authentication and signature acquisition, according to one embodiment;

FIG. 30 depicts an exemplary user interface and system for electronically signing an authenticated document by linking a mobile device such as a smartphone or tablet to a web session while removing the identifying feature such as a QR code once a link has been detected and also providing feedback to the user on the home device as to the type of other device linked to the session to provide guidance to the user of the specific type of device linked, according to one embodiment;

FIGS. 31A and 31B depict an exemplary web user interface and system for capturing an electronic signature through a secondary device that was linked to a web session such as a signature pad or touch screen device including but not limited to a tablet or smartphone such that portions of the signature appear in near real time on a home device or home session as soon as they are entered on the secondary device discounting network lag, according to one embodiment;

FIG. 32 depicts an exemplary web user interface and system for automatic error checking that all relevant information has been completed when documenting advanced care planning or other type of medical form prior to advancement to another page in the session, according to one embodiment;

FIG. 33 depicts an exemplary web user interface and system for linking with a medical record and automatically pulling physician and/or healthcare worker information from that medical record to auto-populate the physician and/or healthcare worker information onto a medical form and an acknowledgement to the process of automatic population of that information to the user, according to one embodiment;

FIG. 34 depicts an exemplary web user interface and system for linking with a medical record and providing signatures for medical documentation through secondary device linking such that once the secondary device is linked once it is allowed to change by directed guidance from a home computer or system which it was linked to in order to allow for multiple signatures to be entered in sequence without requiring separate device linking for each signature capture, according to one embodiment;

FIG. 35 depicts an exemplary user interface and system for capturing an electronic signature image from a secondary device through a web link to a primary device, according to one embodiment;

FIG. 36 depicts an exemplary user interface and system for capturing medical information on a web form including a summary page for review of that information with or without a reminder to confirm the validity of the summary before the document is active, according to one embodiment;

FIG. 37 depicts an exemplary user interface and system for capturing medical information on a web form including a summary page for review of that information before the document is active, according to one embodiment;

FIG. 38 depicts an exemplary user interface and system for capturing medical information on a web form such a POLST document including converting web form populated information into an appropriate document format that can be printed and sent home with a patient as well as stored within an electronic medical record, stored in a cloud, or accessed remotely through a medical record or other secure authenticated access portal, according to one embodiment;

FIG. 39 depicts an exemplary user interface and system for capturing medical information on a web form such a POLST document including converting web form populated information into an appropriate document format that can be printed and sent home with a patient including time and date stamp information for when the form was completed and signed, according to one embodiment;

FIG. 40 depicts an exemplary user interface and system for capturing an electronic signature image for a medical form from a secondary device through a web link to a primary device by taking a photo through a web based QR code reader, according to one embodiment;

FIG. 41 depicts an exemplary user interface and system for capturing an electronic signature image for a medical form from a secondary device through a web link to a primary device by taking a photo through a web based QR code reader that activates the camera on the phone and directs a user to take a photo while also displaying appropriate educational information on correct orientation, according to one embodiment;

FIG. 42 depicts an exemplary user interface and system for completing a medical form via a web interface that is behind an authenticated wall such as single sign on from a medical record system or other secure portal such that a QR code or other linking means is displayed after clicking a “connect to mobile” and/or “sign with mobile” link or button such that this provides a secure way to link a secondary device to the web session for data capture including a signature image, according to one embodiment;

FIG. 43 depicts an exemplary user interface and system for linking a smartphone with a secure web interface by taking a picture of a QR code through a web site or scanning a QR code via a installed app or web app to link the phone to the web session, according to one embodiment;

FIG. 44 depicts an exemplary user interface and system for linking a smartphone or tablet to a web interface for completing forms including medical forms such as POLST while alerting the user as to time to link the devices by showing a compressing image and/or a progress bar on the phone once the QR code or other identifying linking feature has been captured and is being processed by the secondary device such as a smartphone or tablet, according to one embodiment;

FIG. 45 depicts an exemplary user interface and system for completing a web based form including a POLST form or other medical documentation such that once a secondary data device is linked the web session will display identifying information about that device such as its make, or model, or other feature that can guide the user as to its secureness as well as once linked the web session will remove or hide the identifying mark such as a QR code from being able to be scanned by any other devices, according to one embodiment;

FIG. 46 depicts an exemplary user interface displayed on a smartphone or tablet after linking has occurred to the web session such that this interface allows a user to sign a document and it also alerts them to the feature activated on the linked device such as “sign document” “take photo” “scan fingerprint” “shake phone” “press record” “start speaking” “click to begin video” or other phrase or feature that may be desired depending on what type of data acquisition feature has been activated by directions from the web session to the linked mobile or tablet device, according to one embodiment;

FIG. 47 depicts an exemplary user interface and system for capturing and displaying a signature image on a secondary device that is linked to a web session on a primary device that is entered through the touch screen on the secondary device, according to one embodiment;

FIG. 48 depicts an exemplary user interface and system for completing a web form including a POLST or other type of medical and/or advanced care planning form such that a signature image is displayed in near real time and/or real time depending on lag between linked devices on a signature page of a web session displayed on a primary or home device while or shortly after the signature is signed on a secondary device with a touch screen such as a tablet or smartphone, according to one embodiment;

FIG. 49 depicts an exemplary user interface and system for linking a mobile device such as a tablet or smartphone to another device via a web session including an indicator feature that a session has timed out, been disconnected, or is no longer linked to the home device through the web session, according to one embodiment;

FIGS. 50A-50C depict exemplary user interface and system for linking a mobile device such as a tablet or smartphone to another device via a web session including passing information from the another device to the tablet or smartphone such as a video to be played on the tablet or smartphone. This includes the phone following the activity of which section is currently active on the computer terminal such that a specific page must be opened in order to allow for the specific smartphone feature for that page to be active, according to one embodiment;

FIGS. 51A and 51B depict an exemplary user interface and system for linking a mobile device such as a tablet or smartphone to another device via a web session including passing information from the another device to the tablet or smartphone such as a form interface system without identifying information that would cause information entered on the smartphone or tablet linked device to be constituted as identified HIPAA information but instead the identifiers and the information entered on the smartphone or tablet linked devices is otherwise sent together to the system and stored securely and may be accessed in its entirety on the secure another device such that information can be entered and then sent to the system which may also securely record the actions taken on the linked devices to create an audit tracking event of activity recorded in the secure system on the activities of the unsecure interface on the tablet or smartphone, according to one embodiment;

FIGS. 52A and 52B depict an exemplary user interface and system for linking a mobile device such as a tablet or smartphone to another device via a web session by clicking a section of the another device user interface to display a linking code which is temporary in that the linking code would be entered on a web page or app window opened on the tablet or smartphone to link to the specific web session, according to one embodiment; and

FIGS. 53A and 53B depict an exemplary user interface and system for linking a mobile device such as a tablet or smartphone to another device via a web session first using a QR code or other linking system to link a mobile device to a user's account session and/or prompting the instillation of a security certificate, cookie, or other identifying token or password to allow the mobile device to automatically link to the system running on the another device when the another device terminal has been logged into such that a user can then leave the area with their mobile device that is actively transmitting data to the medical record such as when the electrocardiogram is activated, according to one embodiment.

### DETAILED DESCRIPTION

The following description of various embodiments should not be interpreted as limiting the scope of the invention described in the claims. The drawings and descriptions set forth herein should be regarded as illustrative in nature and not restrictive.

Medical documentation is typically entered in on paper, and then that paper is scanned into the medical record and displayed as a scanned document. In certain implementations, the document may be in the Portable Document Format (PDF). Optical character recognition (OCR) and other methods can be used to extract information from the scanned document on a limited basis and display it within a medical record, but such methods for extracting information are very limited. There exists a large unmet need, especially in the areas of advanced care planning and the requirement to provide medical documentation through care transitions, to be able to provide information in a real time way within a single access point with a medical record system. This information may need to not only be entered at the current medical record system but also may need to be able to be entered at any medical record systems and/or another secure portal if desired for healthcare practitioners or emergency personnel that do not have readily available access to electronic medical record systems.

Many types of medical/healthcare related forms must be entered in an electronic, web based format, and many such forms must be signed by a patient, healthcare provider or other user. Just one category of such forms is advanced care planning documentation, including but not limited to POLST, MOLST (Medical Orders for Life-Sustaining Treatment), MOST (Medical Orders for Scope of Treatment), POST (Physician Orders for Scope of Treatment), Advanced Directives, Living Wills, Healthcare Proxy, Power of Attorney, and the like. The embodiments described herein provide a system for completion of these and other types of documents, such as consent forms, birth plans, intake forms, and other medical forms, in a way that provides for authenticated access and completion with signature images and other rich media elements. The system allows for the information from the forms to be in an electronic format that is easily transmittable to other systems of the medical record, which do not communicate easily using currently available systems. The system is not limited to medical form documentation, but may also be used in real estate transactions, business transactions, banking, finance, stocks, legal efforts, ballot initiatives, statute creation, or any other type of documentation where it would be advantageous to complete a document online with additionally robust security, education, and documentation advances, such as but not limited to electronic signature or other data acquisition through linking of at least two devices.

In the case of a healthcare provider, in one embodiment, a user may log in, separate from the medical record system, via a secure portal, by providing demographic information, such as but not limited to their name, date of birth, address, social security number, phone number, license number, DEA number, current location address, credit card number, and/or any other information that would be deemed necessary to indicate that the user is a physician or the desired type of individual to authenticate, without requiring them to complete a separate unique log in for the system and/or register for the system. This may necessitate linking to non-public databases, such as a state health department, the social security administration, or others. In addition, this information can be augmented by data capture abilities from a linked mobile device, such as data captured as a GPS location, IP address, or other means from the phone, including but not limited to data collected from a camera for facial recognition, fingerprint scanner, or audio recorder for voice recognition. Also, in some embodiments, a linked mobile device may be used to scan a form of identification, such as a credit card or driver's license, by either taking a picture of identifying features, including but not limited to a face or digital watermark or barcode, but also may scan or photograph in succession a magnetic stripe or other form of identification or validating mechanism like a hologram. The combination of successive data elements corroborated between entered and captured information and/or entered information and third party or primary databases will authenticate the user without requiring them to have a unique log-in or user name and password that they create once and then enter each time they want to log in. This is an optional feature, however, and some embodiments may be able to identify a user that has unique credentials, such as a physician, by simply knowing select information, such as a DEA number and/or authenticating a digital watermark on their driver's license that is not publicly available.

In the event that a user is already in a system that is linked to the document management, completion, and access system described herein, then the system that is linked to the present system may serve as the authentication for the user. In this case, such as with an electronic medical record system, a user would sign into the medical record system with their user name and password. Once the user has signed in, a secure link may display an access portal to the web based system described herein. This access portal may simply need to be accessed from inside an already authenticated electronic medical record system for the user to gain access in a secure way to the described system to complete and/or access documentation.

In some embodiments, for example, a hyperlink may be displayed on a medical record chart of an electronic medical record system. The link may be preloaded with the access provider ID, the patient medical record number and a private shared key to encrypt the link. The link text may also be hidden. Once the link is clicked, it validates the key and decrypts the link and then loads the proper patient. The key may serve to decrypt URL parameters. When the link is clicked, the user is provided with a webpage, and the webpage decrypts the parameters and uses them to look up the provider and patient in the database. If neither is found, then the webpage displays an error or redirect message. The database that is used to check for the patient is populated with data from an HL7 (Health Level 7) feed from the healthcare institution. If a patient record is not found in that database populated from that HL7 feed, then the webpage redirects to the same error or redirect message. The patient should be found, because an HL7 event message is triggered when a patient is admitted or another action happens. If the patient is not found, then that means the decryption somehow redirected toward a patient that does not exist. The system may include built-in security to check the database as a measure to prevent unauthorized access. The parameters are sent from the webpage to the back end for decryption, and then they are used to search the database. If the parameters are not found in the provider database, then the system may provide view-only access or may deny access, depending on the preference of the healthcare institution.

If the provider is found then the system may allow the correct access controls, based on the specific type of provider accessing the system. When the patient is found in the parameter search in the database, then the system may return the patient record from that parameter that was in the database. This may also include a matching lookup. This matching lookup may take a parameter and search our database to find the HL7 message that was sent from the same site that sent the parameter. The HL7 messages may be sent through a video phone to populate the database for searching. Then, once the parameter for the patient has been found in an HL7 message, the system may use the rest of the data present in that HL7 message or other HL7 messages from that parameter number to pull up the demographic or healthcare identifiers on that patient. The system may then use the demographic or healthcare identifiers with a records matching algorithm to search for other records within a database that match those identifiers but that are not part of that original record from that single site that the access came from. This allows the system to link multiple records together. This matching system may be done in batch prior to the parameter from one site coming in or in real time. Once the demographics match and all the records are matched, then the full record or a partial record is displayed on the webpage for access and interaction from the provider in the medical record.

To complete more sensitive documentation, such as a physician orders for life sustaining treatment (POLST) form, it may be desired to provide just in time educational media, such as photos, videos, audio files, or other means to a patient, while a healthcare provider is filling out the documentation on a web based platform. This information, its playing or lack thereof, including the tracking of mouse clicks and movements or any other activity, could be tracked via an audit tracker feature. This audit tracking can then be converted into a media element or summary report that is attached to the POLST form, so that an accessing provider at a future session may then use it, in conjunction with the POLST form, to make a medical decision about the form.

Additionally, some users and/or patients may want to share their form with loved ones or family members. A “Share my POLST” or “Share my Proxy” or “Share my Advanced Directive” or “Share my Living Will” feature at the end of the session may be provided, so the patient, provider, or other individual with sharing authority may elect to enter sharing information, such as email addresses, physical addresses, phone number, or other information as to how the entered documentation can be sent to the desired party. This includes but is not limited to the document of the form created or generated, as well as all audit tracking features and media elements.

In some embodiments, if a patient wants to cancel his POLST form, he can call a number at the bottom of the POLST form and enter a unique set of numbers to identify himself. Additionally, he may call his healthcare provider, who can take verbal consent over the phone and indicate such on the POLST form in the medical record, which will track this in the audit log. In various embodiments, this described workflow may be used for any type of documentation, and the example of a POLST form is provided here for exemplary purposes only and should not be interpreted to limit the scope of the invention.

In the event that the linking of a secondary device for signature capture is not possible, a form can be completed in an electronic format, absent signatures. Then, in order to get the signature, the form would be printed with an identifying QR code unique to the web session that the form was printed from. QR Codes are machine-readable optical codes that can be used to store information. While reference may be made to QR codes throughout this specification, a person of skill in the art would understand that other kinds of machine-readable codes can be used, including but not limited to bar codes, matrix codes, and other formats. Then, the patient, provider, or any other user or person that should sign a particular form can do so. Then, the document can be faxed to a computer system that reads the QR code and files the signed copy of the document that was physically signed with the identifying information entered in the web session to create a complete record. Prior to the entry of that type of physical form via fax, scan, or other method of entering in an image of the form with an identifying mark such as a QR code, the web session may display “form out for signature” or “awaiting scanned copy” or “awaiting faxed copy” or “awaiting signature,” such that an accessing party may know to contact an individual for more information. This type of documentation process to create an electronic record while also allowing for a physical hard copy signature may be especially relevant to notary public authentication and/or to the process of requiring witnesses on forms where the web form is populated and printed with an identifying mark or feature that can be read by the computer once it is scanned, faxed, and/or uploaded to the system and that action of digitizing an image of the physically signed form allows the system to link the form to the electronically entered information. The use of this method may be especially useful for advanced directives and healthcare proxy forms, but is not limited to such forms.

Sometimes it may be desired to display information on a different device platform, such as a tablet or smartphone. However, these devices are different, and many of them are personal devices owned by healthcare workers or patients. Furthermore, tablet and mobile devices owned by an enterprise may require additional time consuming configuration to be able to manage secure, private and confidential data, for example Protected Health Information. It then becomes necessary to secure these devices through encryption before opening personal health information on these devices. This many not be practical in many settings. One such use of disclosed embodiments may be to link a tablet or smartphone to a secure system running on a computer terminal or other device such as an electronic medical record. This linking may occur through scanning a QR code, near field detection, inputted code, BLUETOOTH, WI-FI, RFID, or any other means. Once the link is established, the tablet or smartphone device would then be linked, such as it would have an app and/or web session running on it that reflects activities in the secure system that the secure system allows of it. Some uses for this may be to link the device such that signatures may be captured on the tablet or smartphone and then sent to the computer and in that case a signature pad feature would be pushed to the smartphone or tablet for user interaction. In a further exemplary embodiment, a video may be pushed to that smartphone or tablet such that it could be displayed in a more user friendly way and then watched. Any activities on the phone, such as playing, stopping, or pausing the video, would be recorded by the audit tracking tool on the system displayed on the other device displaying the linking to the secure system. In this case, no personal health information need be disclosed on the mobile device, because it is all stored on the system, which does not grant full access to the mobile workstation. The system is able to combine the data gained from interactions with the mobile workstation to that of information in the system database and entered into on the workstation originally displaying the ability to link such as a desktop computer. This complete record is stored securely and is personal health information, but the data entered into on the phone or tablet that was linked to the system is generic and need not be identified or linked to personally identifying information running on the phone or tablet. The advantages of this type of system is that there is no risk of HIPAA breach or disclosure of personal health information on the smartphone or tablet when accessing generic data information pushed from a system to be interfaced with the patient or healthcare provider on the mobile terminal.

Another potential use of the system described herein is to link a mobile device to a terminal displaying a system running in a secure environment. In this event, an entire form set, with or without personal identifiers, is pushed over to the tablet or smartphone. This allows a form to be completed remotely, such as by selecting choices in different sections of the form and/or entering or typing preferences that may or may not themselves be personal identifiers. Then, as required, because the smartphone or tablet where the information was entered was linked to the medical record, the data from the smartphone or tablet can be combined with personal identifying information stored in the system or medical record and accessed via a secure terminal in order to create a full record. This feature may also extend beyond form completion to diagnostic devices such as electrocardiograms, EEGs, dialysis devices, drug packaging label scanners, ventilators, pulse oximeters, microchip counters on pills, electronic drug delivery devices such as inhalers or injector pens, electronic pill bottles, glucose sensors, infra-red sensors, or any other device that may interface or connect to a system or device or itself be capable of linking to a secure session, for example through a QR code, to send data to a secure system and only receive non-personal health identifying information in return. This may also extend to a patient's smartphone or tablet or other device that is linked to the computer system running the electronic medical record. In this case and in others it may be useful to use security certificates to more strongly establish a more long term connection or validation protocol, such that the link would remain active to that specific patient record or user account, even after the patient and the linked device is no longer in the vicinity of the linked system and device, such as outside of the hospital.

In order to link a smartphone or tablet, it may be desired to do so in a manner where a code retrieved from a secure area of the system is entered on the smartphone or tablet. In this session, a user would click a “connect to mobile” button, which would display a code that can be entered on the smartphone. This code may need to refresh every time the user clicks the connect-to-mobile link. Then the user would view the code on the secure environment and enter that into the mobile website accessed through a mobile phone or tablet or even another desktop session. Once the code is entered, that device sends a signal that it desires to be linked. The session may link, or there may be a need for a second code to be displayed on the mobile device that would be entered on the desktop. This secondary authentication feature may not be necessary but may enhance security. Alternatively the user of the secure session would be prompted “would you like to link a mobile device,” which may include an identifier such as GPS location or the type of device. Once the user verifies that the link is OK, then that may also be another means of secondary device authentication.

Another exemplary user interface and system for linking a mobile device, such as a tablet or smartphone, to another device via a web session may include the use of a security certificate or other identifying token or password, such that the link only has to occur once at the physical location to verify the device has privileges to link. This may first occur using a QR code or other linking system to link a mobile device to a user's account session and/or prompting the instillation of a security certificate, cookie, or other identifying token or password to allow the mobile device to automatically link to the system running on the another device when the another device terminal has been logged into.

Once devices are linked using the system currently disclosed, either or both devices may be used for data acquisition purposes, including but not limited to keyboard, touchpad, mouse, microphone, camera, GPS, and gyroscope. Similarly, either or both devices may be configured to display various components of a web page, web form, movie, video, audio recording or other media, text or data that is desired. Such displays may be optimized for tablet, mobile device or desktop. For example, if a web page is displayed on the web session of a desktop computer or other device, and a mobile device is linked to the web session, such linking may cause a portion of the web page to instead be displayed on the mobile device.

Data entry on one device may or may not be populated in real time on the other device's web session. Similarly, data entry on one device may modify or cause to be updated a layout, configuration or other data displayed on the other device. Data entered on either device may be stored on a central server or cloud.

Although the current system may be considered as a way to link two web sessions together, similar linking to facilitate joint or shared data acquisition and display may be achieved through native or web applications designed for specific device(s) and its (their) associated operating system(s), in order to achieve the same functionality described herein.

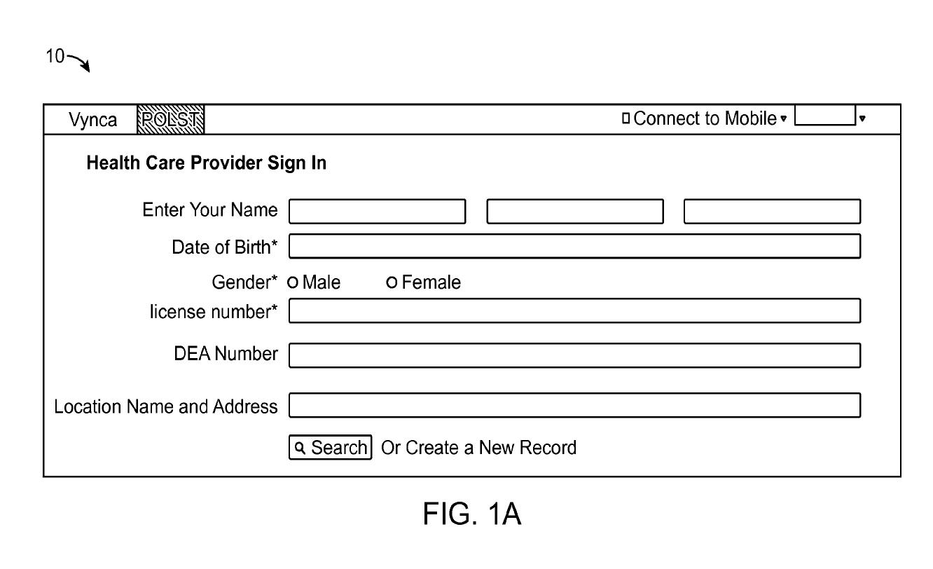

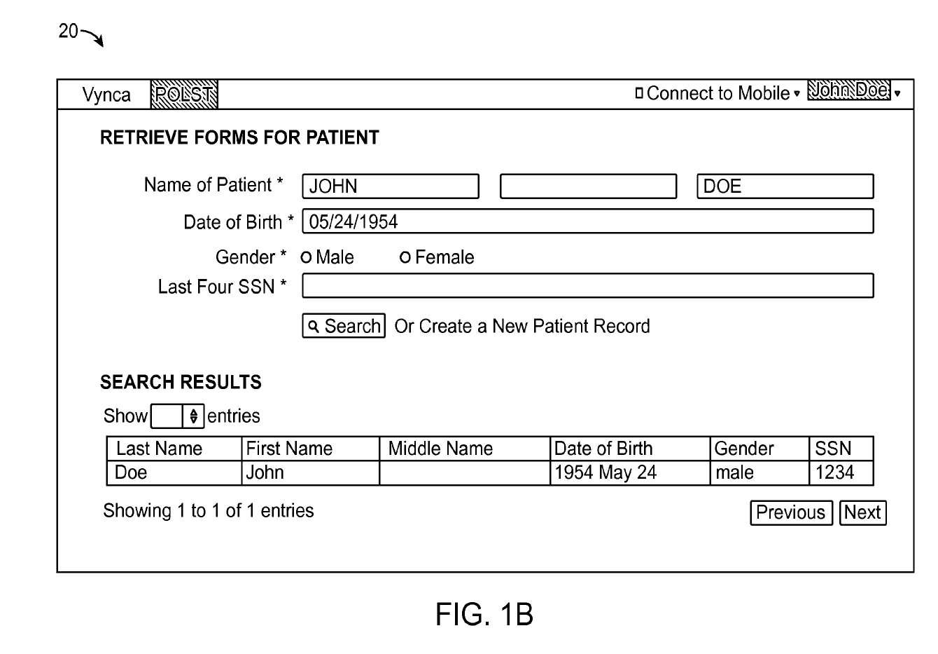

FIGS. 1A-5 are various screen shots illustrating a system for accessing patient information, according to one embodiment. In many of the screen shots provided herein, the system is labeled “Vynca.” This is simply an illustrative name for the system and should not be interpreted as limiting. For the purposes of this application, the terms “Vynca” and “the system” are interchangeable, but neither term should be interpreted as limiting the scope of the other or of the claims of the present application. More specifically, FIGS. 1A-5 illustrate one embodiment of a user interface (UI) that the system may provide inside an electronic medical record (EMR). (Note that the EMR border is not shown in FIGS. 1A-5.) These figures illustrate a method for completing a Physician Orders for Life Sustaining Treatment (POLST) form, according to one embodiment, including a % completion bar, an identifier that shows which state's form is being completed, varying levels of access to historical or older forms, audit tracking records, and a summary page. In general, FIGS. 1A-5 illustrate a method for linking and auto-displaying to the user to the presence of an electronic medical record in a cloud based system that is accessible through a single sign-on portal that passes over the unique access ID and the unique patient ID when a link is clicked, which allows the system to return the record.

FIG. 1A depicts a screen shot 10 of a healthcare provider log-in page, according to one embodiment. In this embodiment, one or more of a license number, DEA number, unique location and IP address match, mobile phone linking with GPS, demographic information and name, or other method of identification, provided together or separately, are used to authenticate a specialized person, such as a healthcare provider, for remote login to a system through a web portal for entering secure information such as healthcare information. FIG. 1B depicts a screen shot 20 of a patient form retrieval page of a user interface and system, according to one embodiment. In this embodiment, the retrieval page may be used for retrieving a patient form or other user form by inputting specific demographic information about that person through a secure interface behind a remote log in authentication station or a single sign on access through an authenticated user location, such as a medical record.

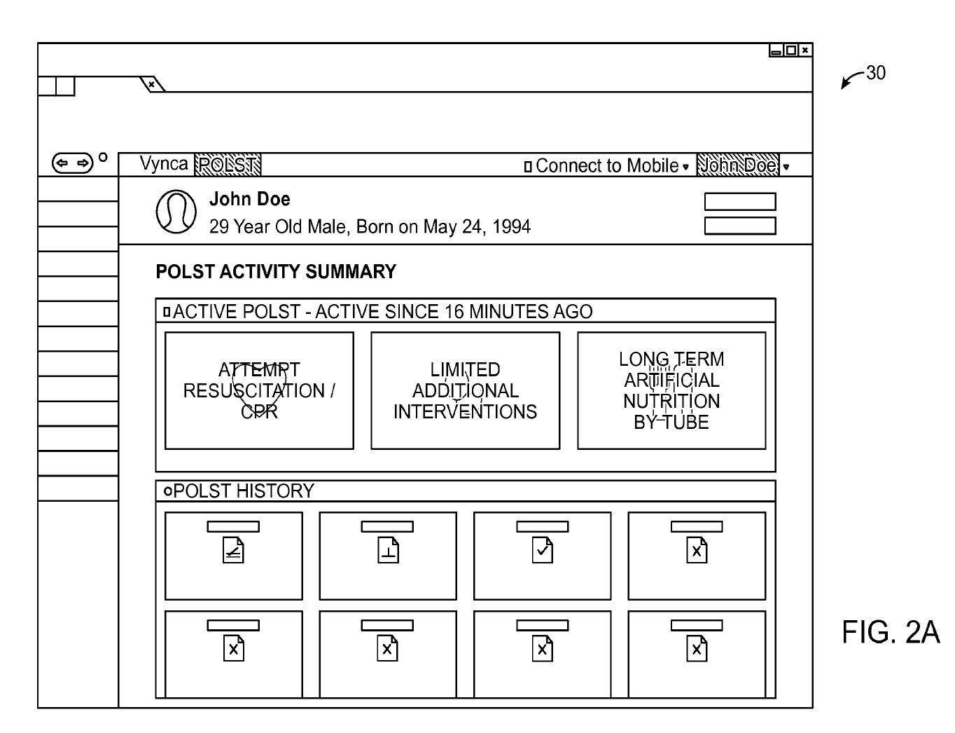

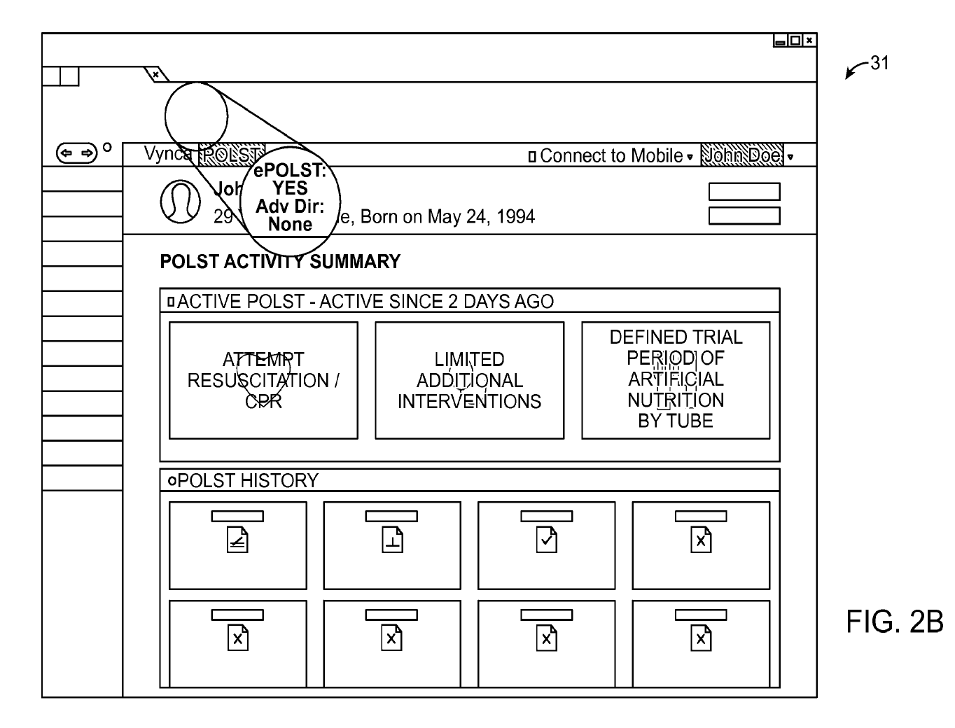

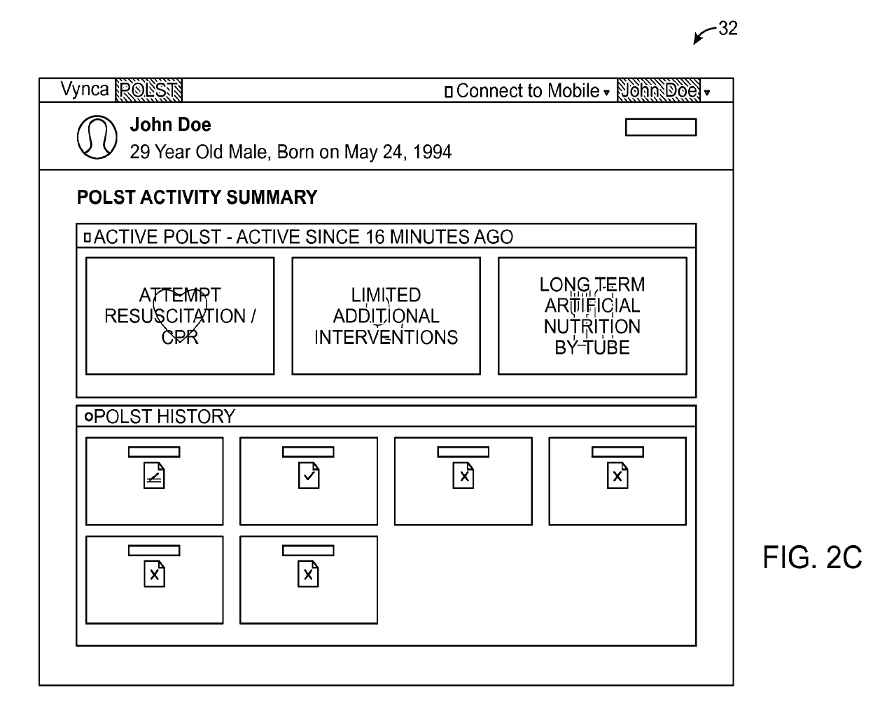

FIG. 2A depicts a screen shot 30 of an activity dashboard page for POLST of an exemplary user interface and system, according to one embodiment. This screen shot 30 depicts an activity dashboard as it may appear within an electronic medical record system. FIG. 2B depicts a screen shot 31 of the same activity dashboard but illustrating a yes/no feature used to identify an advance directive or a POLST form or other Advance Care Planning Document in the system. FIG. 2C depicts a screen shot 32 of an activity summary page for POLST. The activity summary page may be used for identifying information in a system that was previously entered about an individual by a user, such as a healthcare provider entering in POLST documentation including a historical log of such information and audit tracking.

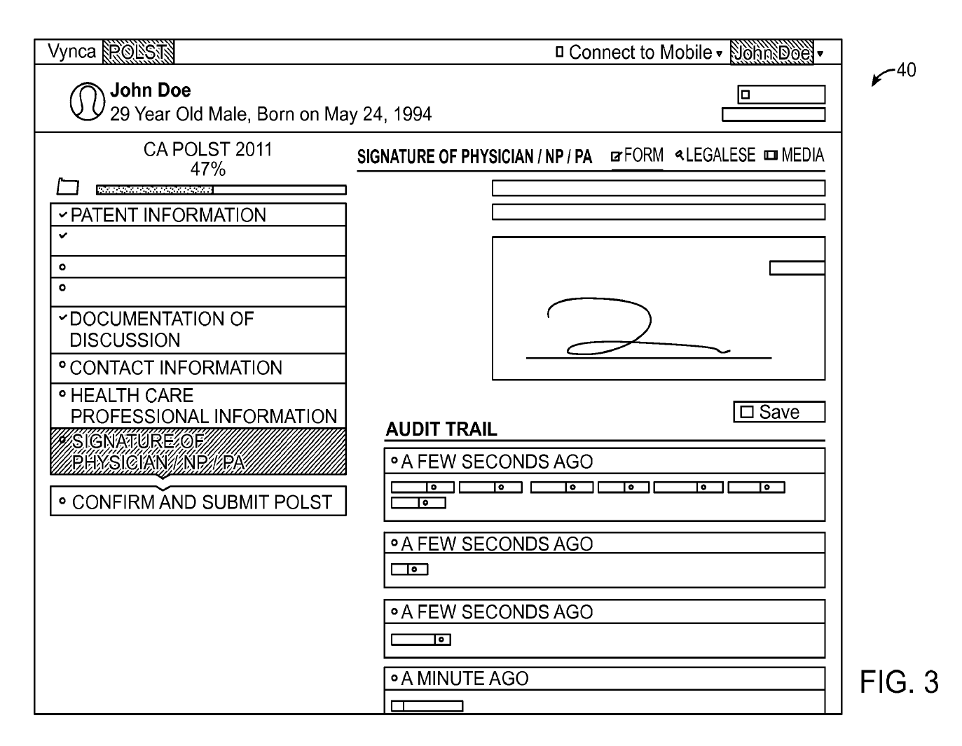

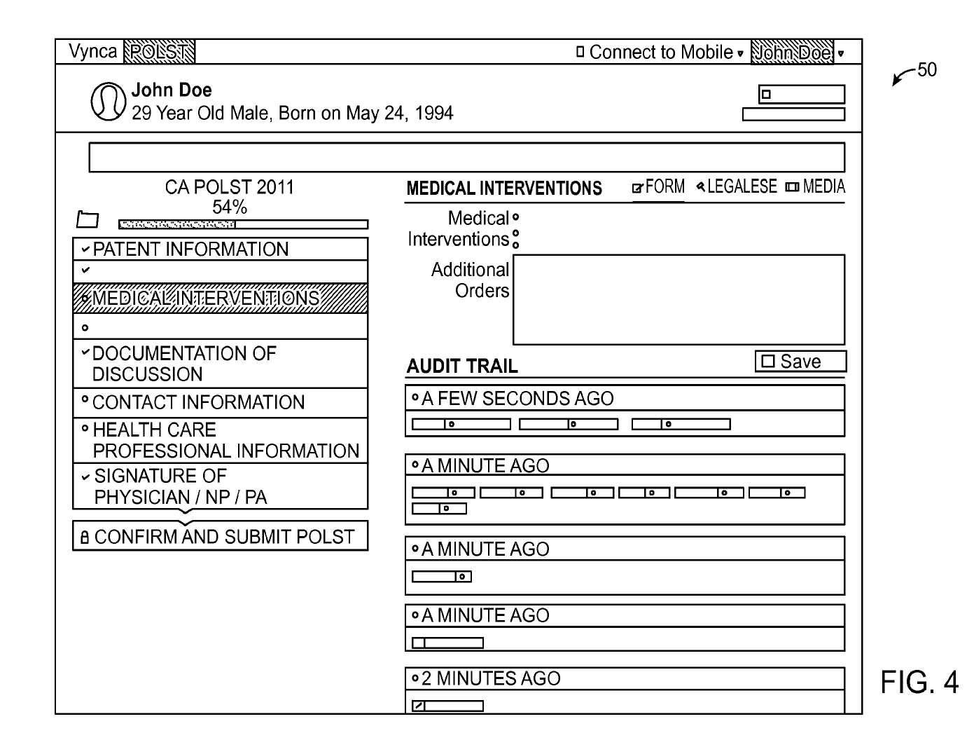

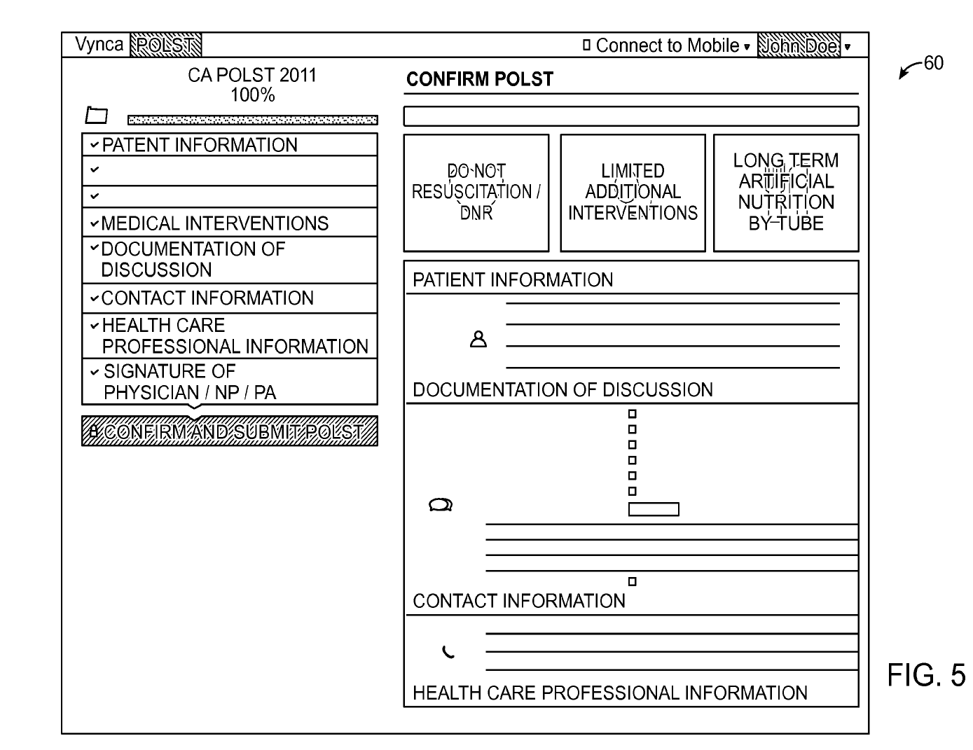

FIG. 3 depicts a screen shot 40 of an electronic signature page of a user interface and system, according to one embodiment. The electronic signature page may be used for capturing an electronic signature image through a user interface for creating a sensitive and authenticated document such as a POLST document. FIG. 4 depicts a screen shot 50 of a medical interventions page of a user interface and system, according to one embodiment. This page may be part of an automatic check for form errors in a secure document interface. FIG. 5 depicts a screen shot 60 of an information confirmation page of a user interface and system, according to one embodiment.

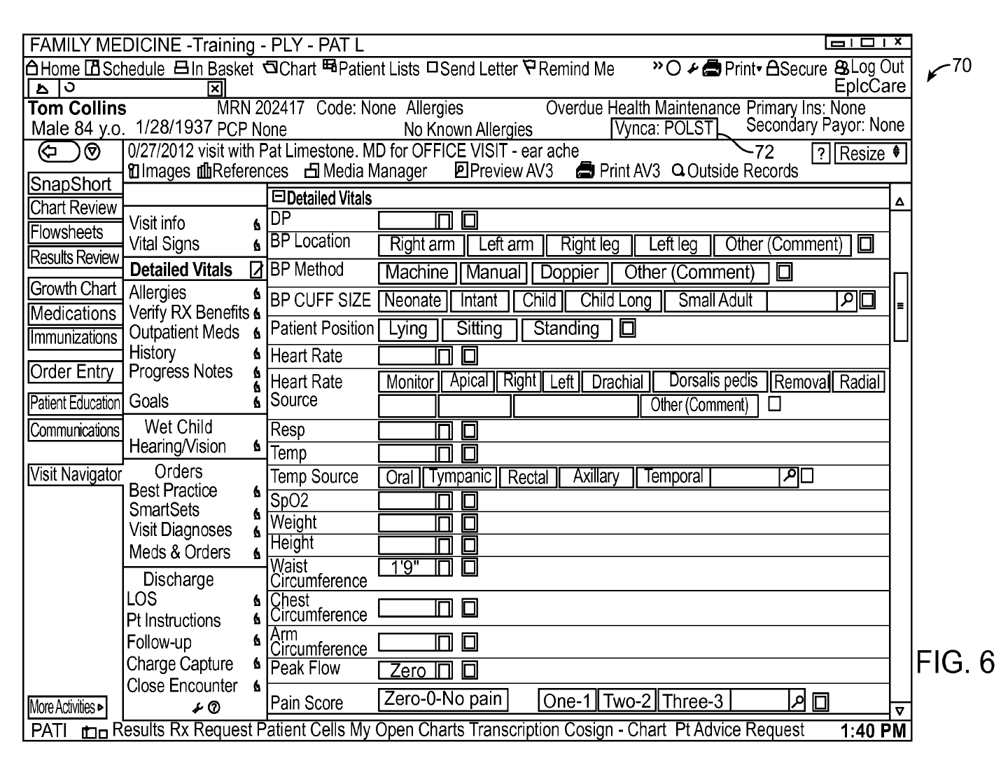

FIGS. 6-9 are screen shots of various pages of an electronic medical records system, illustrating a system for linking to a separate application through an embedded link. More specifically, FIGS. 6-9 illustrate a method of accessing the system and a highlighted link when a record is present. In addition, they show a system that has the advance directive, POLST, and healthcare proxy forms all in one tab that is accessible from the electronic medical record. This embodiment of the system not only provides for linking out to a third party through single sign-on, but also provides a single location of multiple ACP documents as separate links to different document portals.

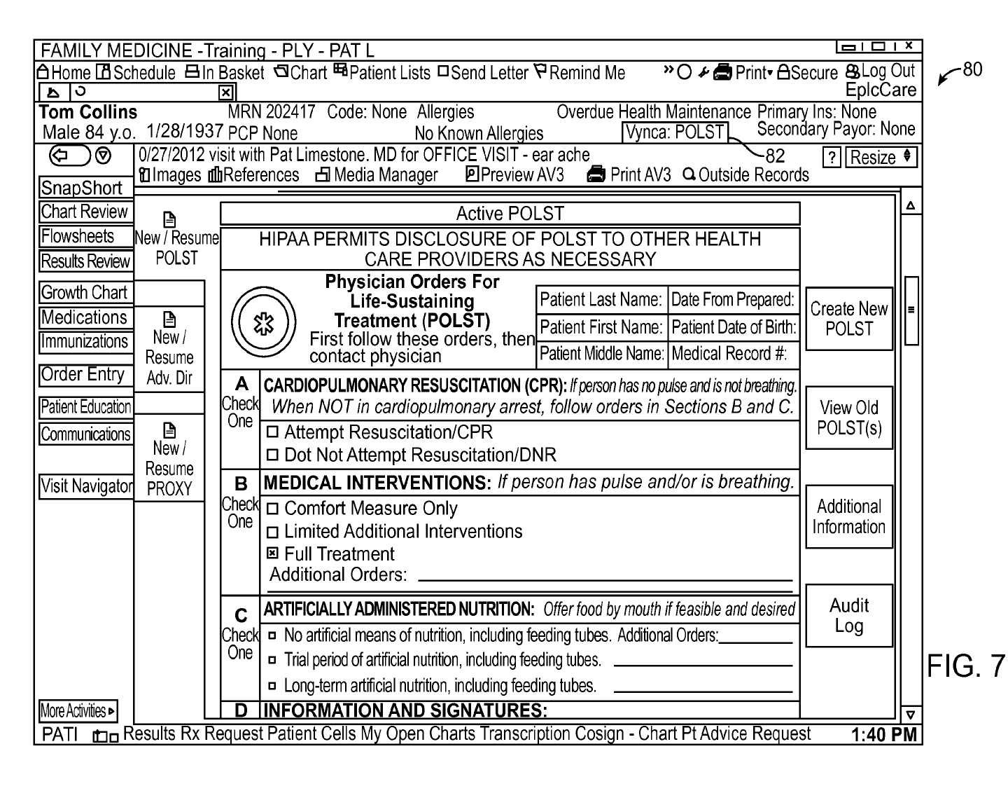

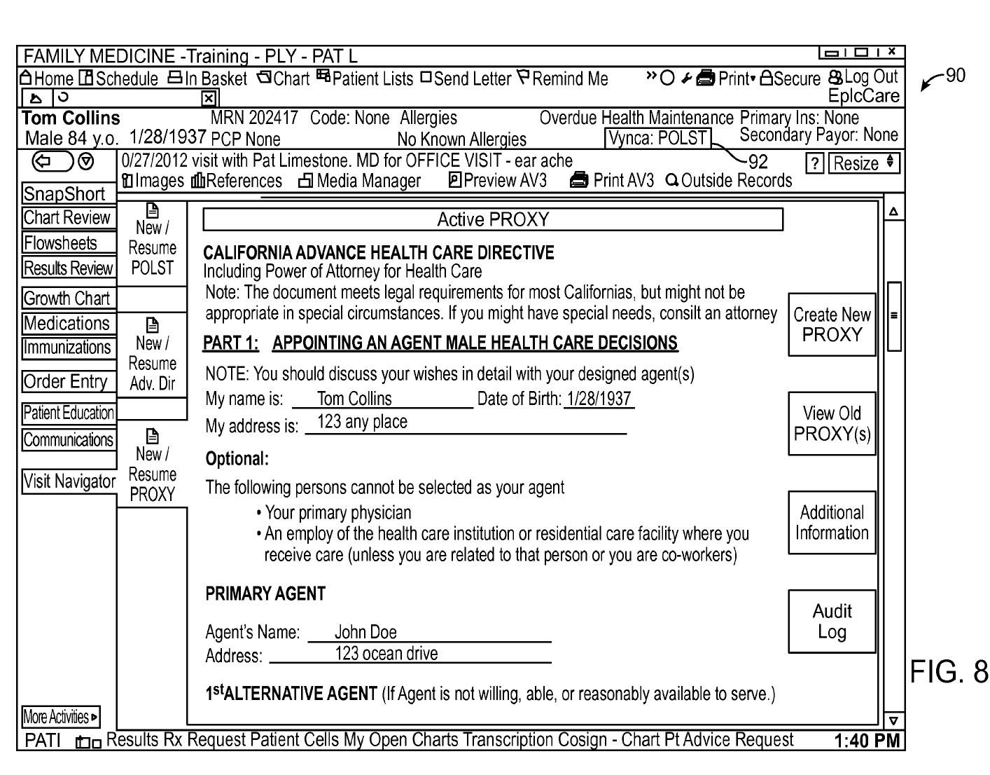

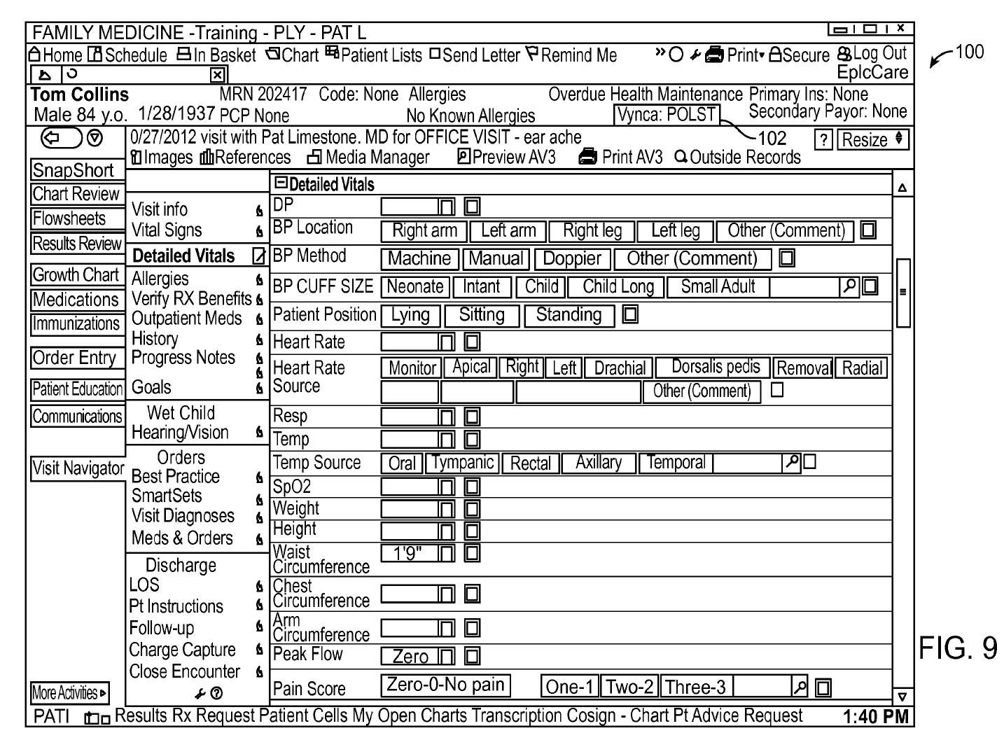

FIG. 6 depicts a screen shot 70 of an exemplary user interface and system for accessing an active medical form, such as a POLST form, from a link 72 or access point within an electronic medical record, including a visual cue prior to clicking the link as to the presence or absence of a document to be accessed from the link. FIG. 7 depicts a screen shot 80 of an exemplary user interface and system for accessing active medical information, such as a POLST form, advanced directive, or health care proxy form, and any audit history or form creation or invalidation history, accessible via a link 82. FIG. 8 depicts a screen shot 90 of an exemplary user interface and system for accessing active medical information such as a healthcare proxy form, including an access point to create a new form, view old forms, view any additional information, or view an audit log of accesses, views, changes, and/or edits, accessible via a link 92. FIG. 9 depicts a screen shot 100 of an exemplary user interface and system for accessing an active medical form, such as a POLST form, from a link 102 or access point within an electronic medical record, including a visual cue, prior to the clicking of the link, as to the presence or absence of a document to be accessed from the link, where in the example shown, no link is presenting a visual clue as to the presence of a form indicating its absence.

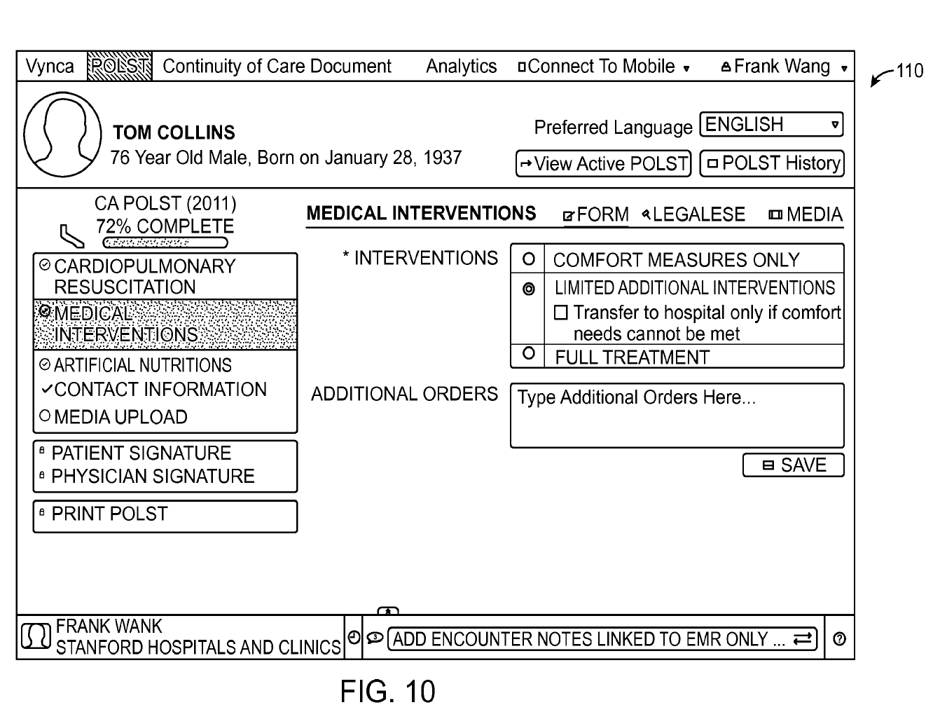

FIGS. 10-14 depict an exemplary user interface and system for entering document completion information. This embodiment of the system includes language translation features, embedded media features, audit log and access features, and note entry capability that is able to be pushed back into the EHR (electronic health record).

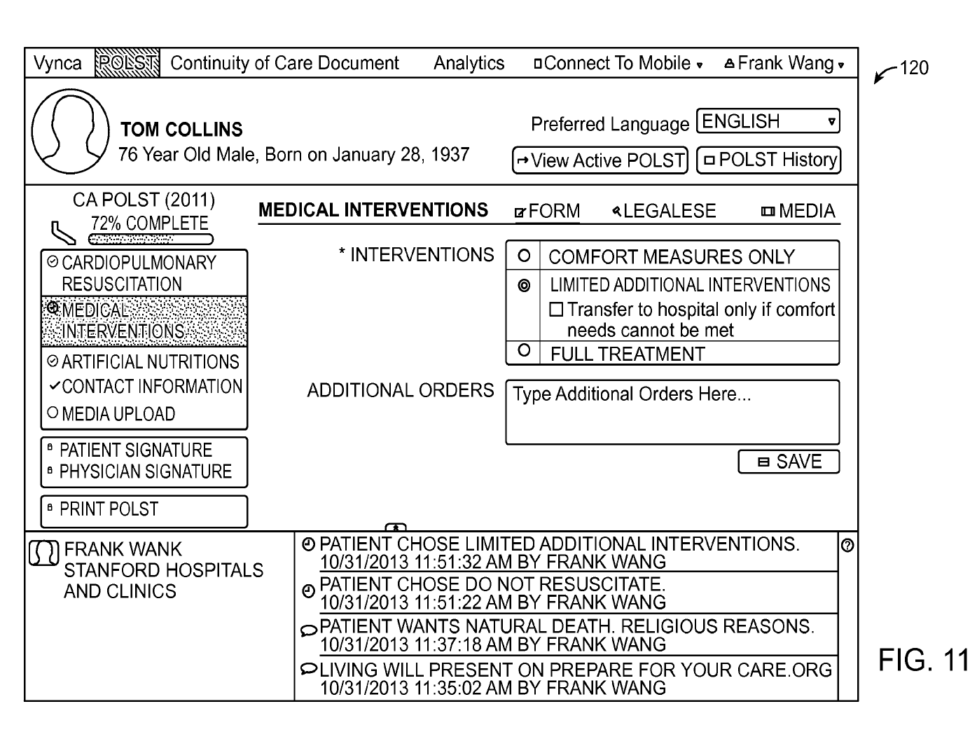

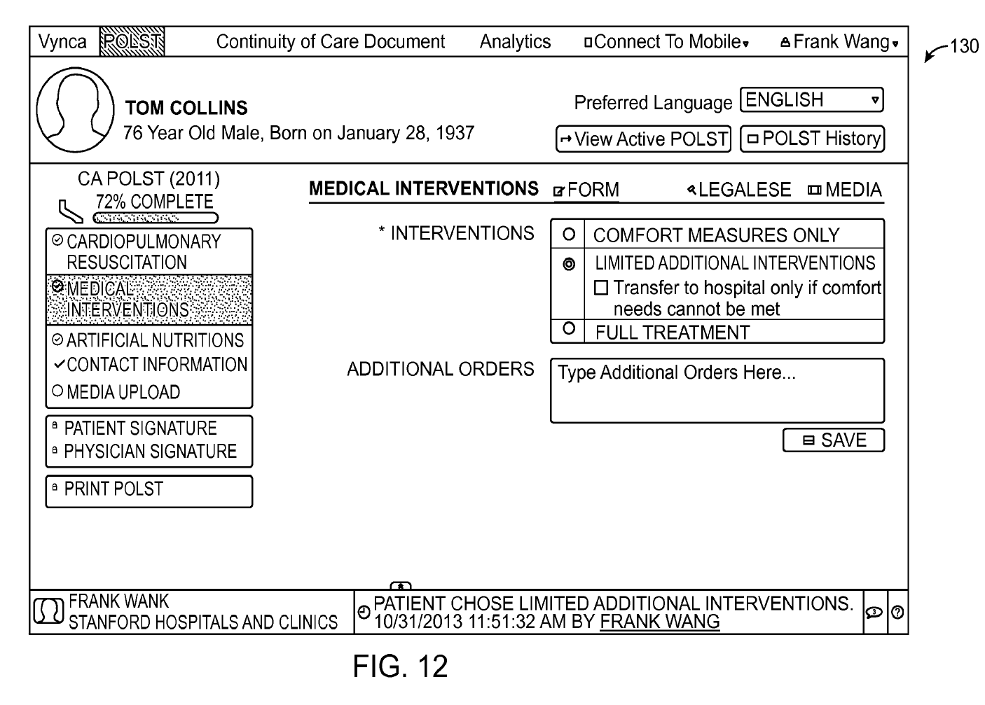

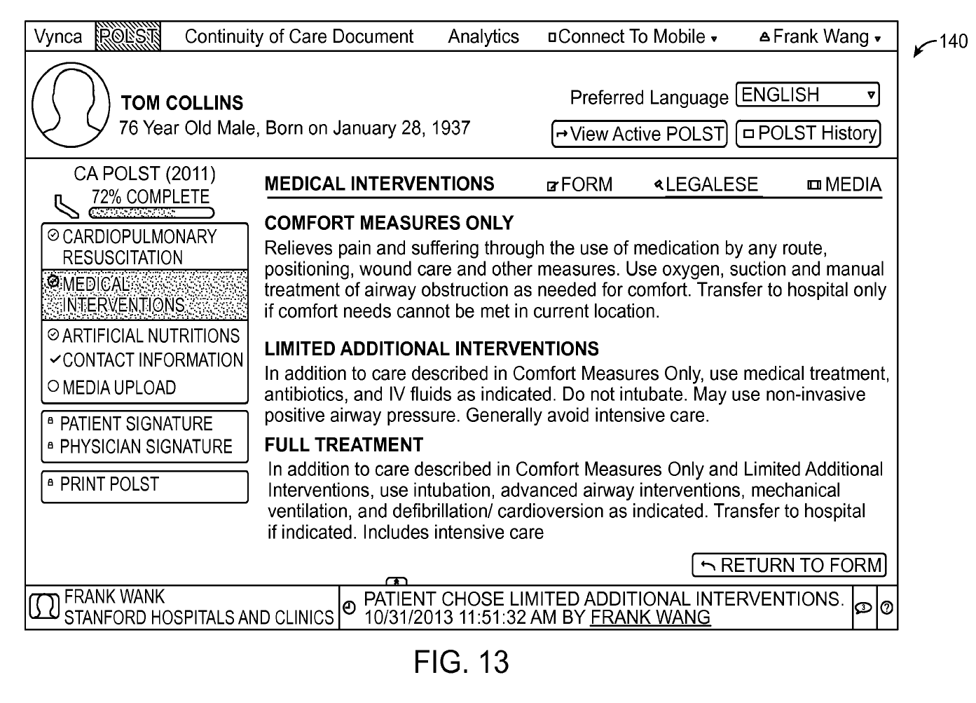

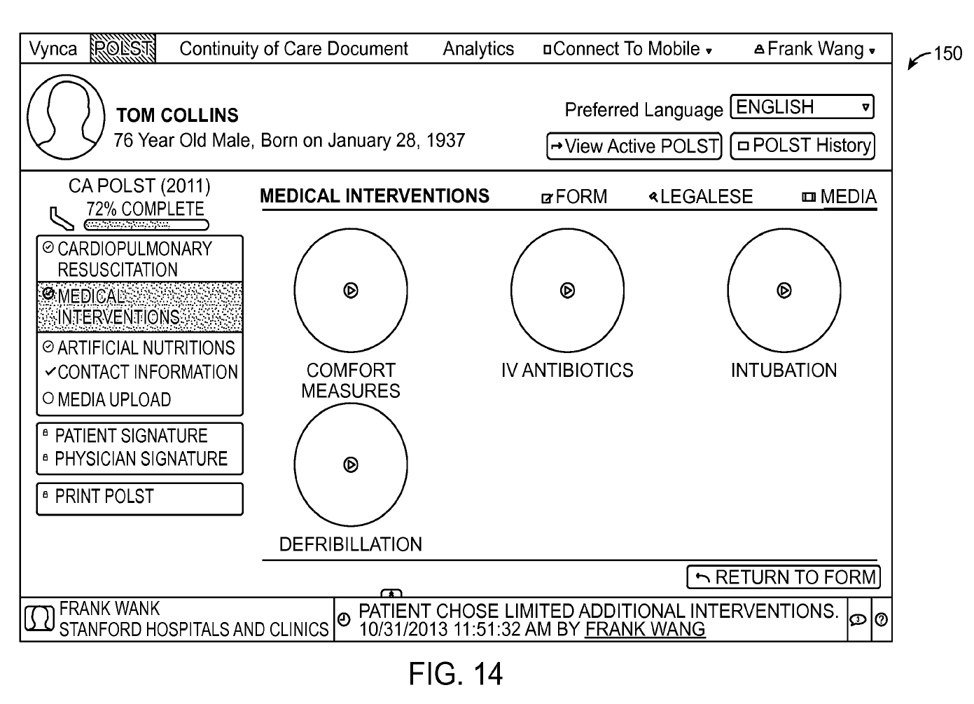

FIG. 10 depicts a screen shot 110 of an exemplary user interface and system for entering document completion information, such as for a POLST or other medical form, including a patient's wishes for desired treatment to be entered by a physician. FIG. 11 depicts a screen shot 120 of an exemplary user interface and system for entering in document completion information such as for a POLST or other medical form while also demonstrating an active audit tracking display 122 to provide report information as to the quality of a conversation taking place when the form is being completed. FIG. 12 depicts a screen shot 130 of an exemplary user interface and system for entering patient information into a medical form tool, where medical information can be selected by a physician or patient to demonstrate a patient's choices for care. FIG. 13 depicts a screen shot 140 of an exemplary user interface and system for documenting sensitive information, including a medical form, such that the legal language and/or terminology definitions are displayed within a link about the current active page or session. FIG. 14 depicts a screen shot 150 of an exemplary user interface and system for entering in information for a document electronically while also providing targeted just in time education for critical sections of the form and/or choices required including but not limited to medical interventions and videos or education around those choices as they pertain to advanced care planning documentation, such as a POLST form.

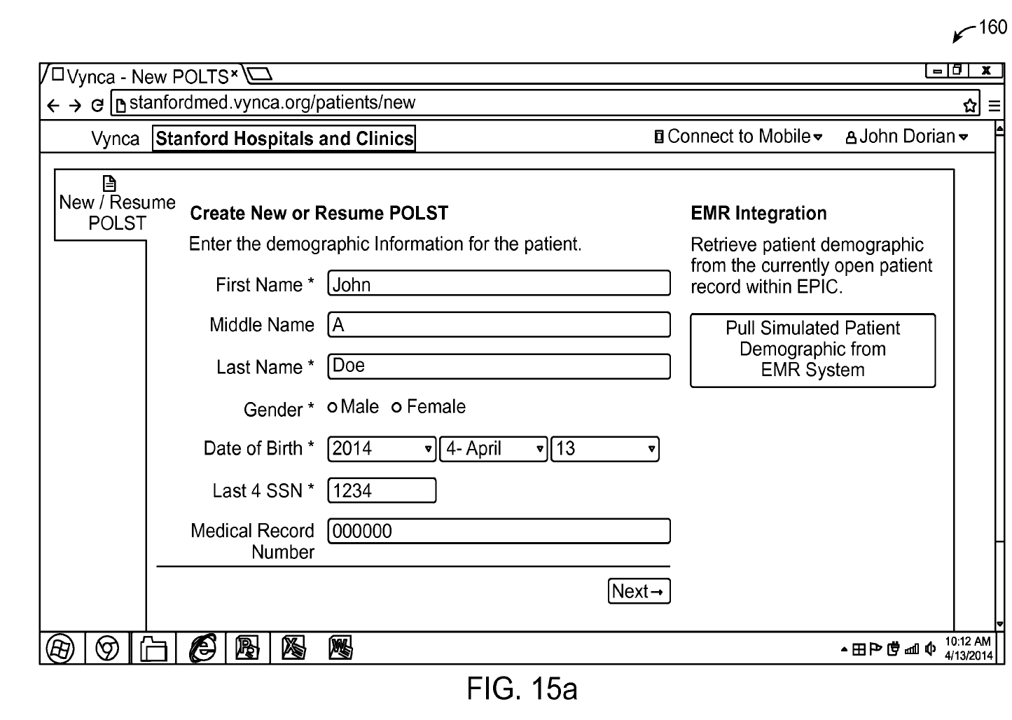

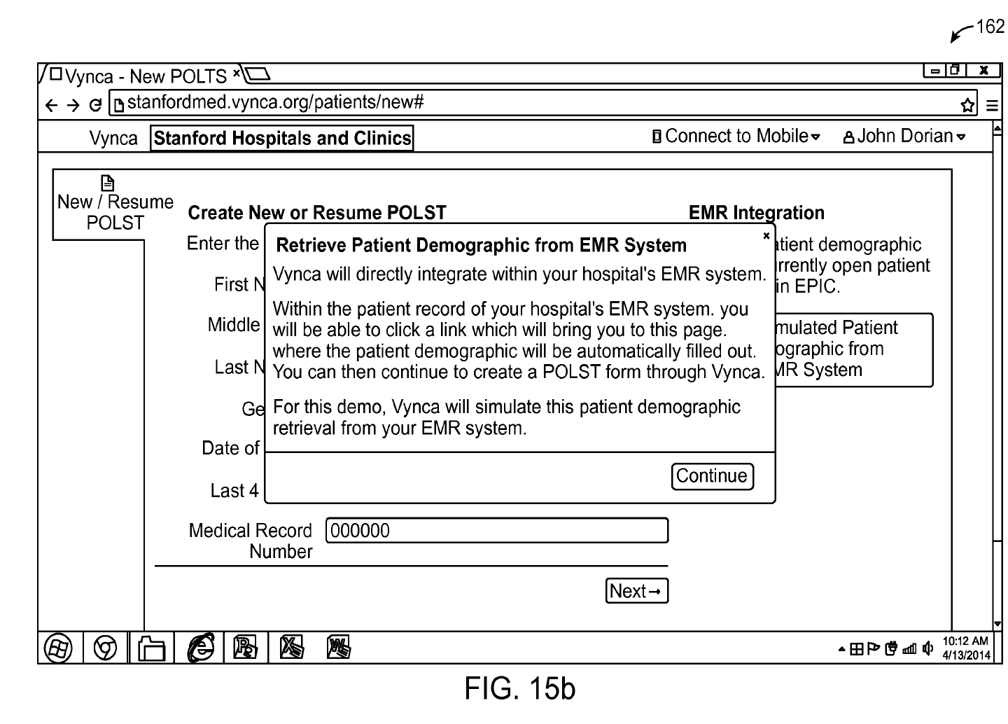

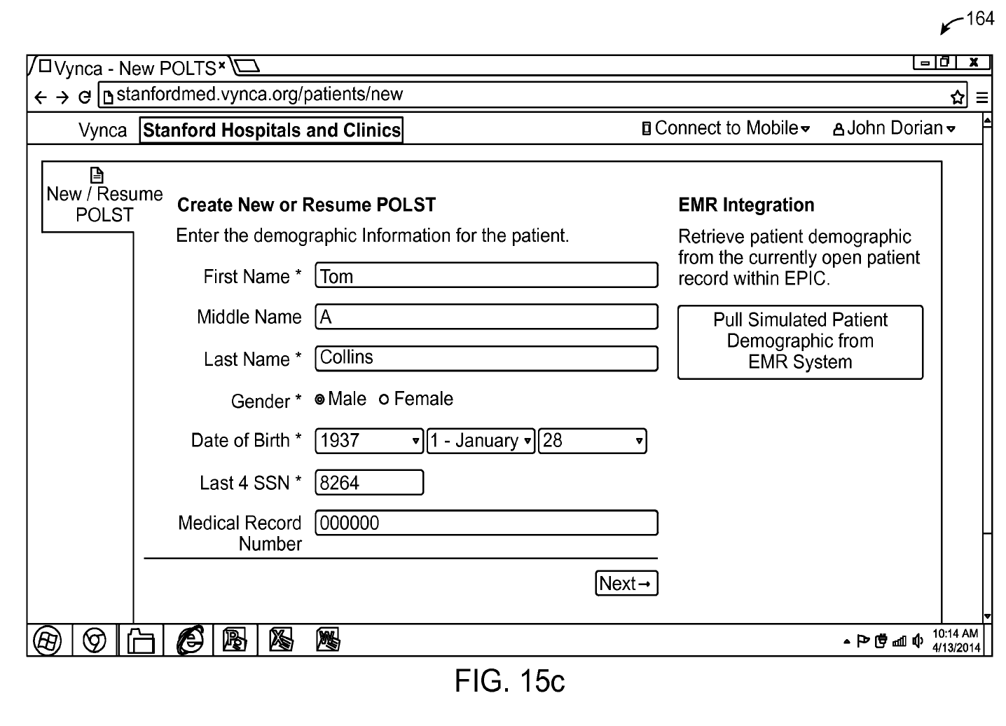

FIGS. 15-42 illustrate one embodiment of a workflow process and features of a system for use with a POLST form. The features include picture capture through mobile device, signature capture through mobile linking, converting of discrete data elements to auto-generate the document with signature, multiple language translations, the ability to backload physician and patient data to the EMR, and signature addition/entry in real time.

FIGS. 15A-15C depict an exemplary user interface and system for documenting information, such as a POLST form, in an electronic form, such that integration with a system, such as an electronic medical record, allows for active pulling of patient and/or provider information into the record and auto-completing of that information within the form for review by the completing party. FIG. 15A is a screen shot 160 of a page for creating a new POLST or resuming a POLST. FIG. 15B is a screen shot 162 of a page for retrieving a patient demographic from an electronic medical records system. FIG. 15C is a screen shot 164 of the page for creating or resuming a POLST, with patient information populated therein.

FIG. 16 depicts a screen shot 170 of an exemplary user interface and system for documenting medical information including information useful for patient identification in an emergency, such as a picture, by linking a smartphone to the web session through secure QR code or other link.

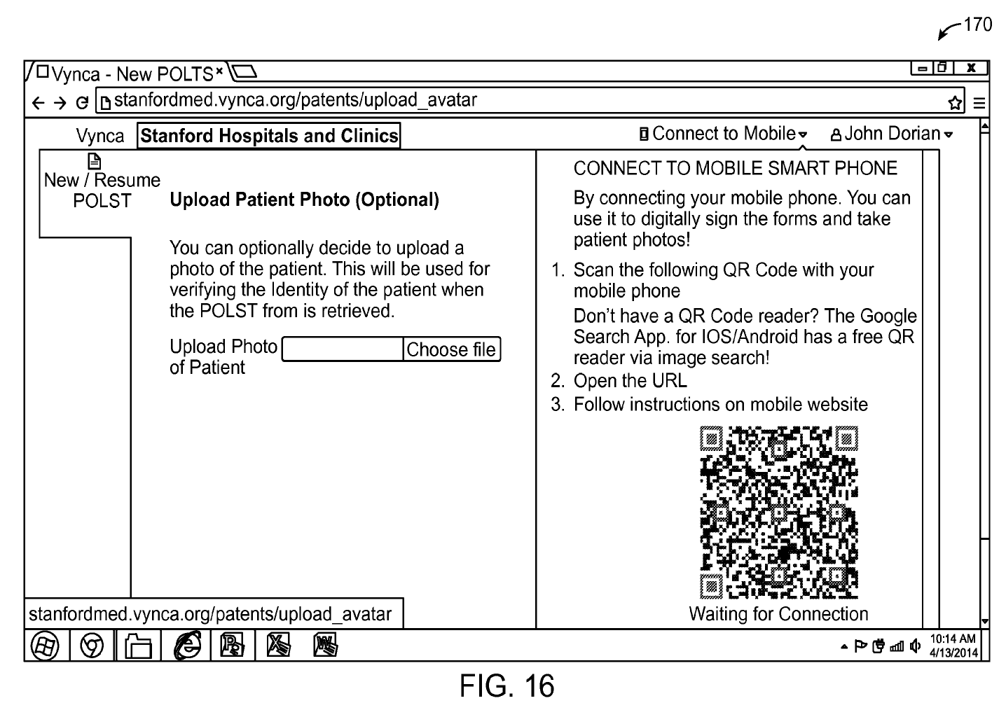

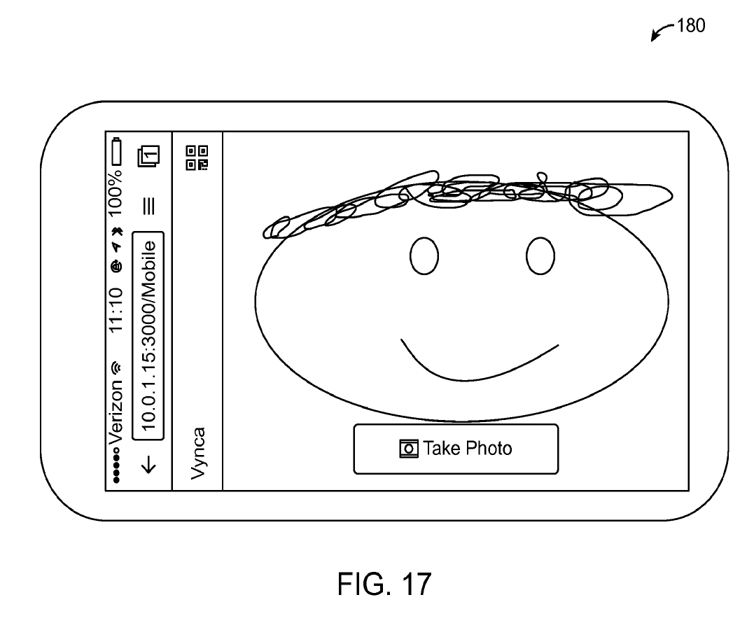

FIG. 17 depicts a smart phone 180 for use with an exemplary user interface and system, for documenting medical information including a picture, by linking the smartphone 180 to the web session through secure QR code or other link and then taking a photo of a patient, which is automatically saved to the web session running both on the phone 180 and/or another system, such that the picture image is not saved directly onto the hard drive of the smartphone 180 itself.

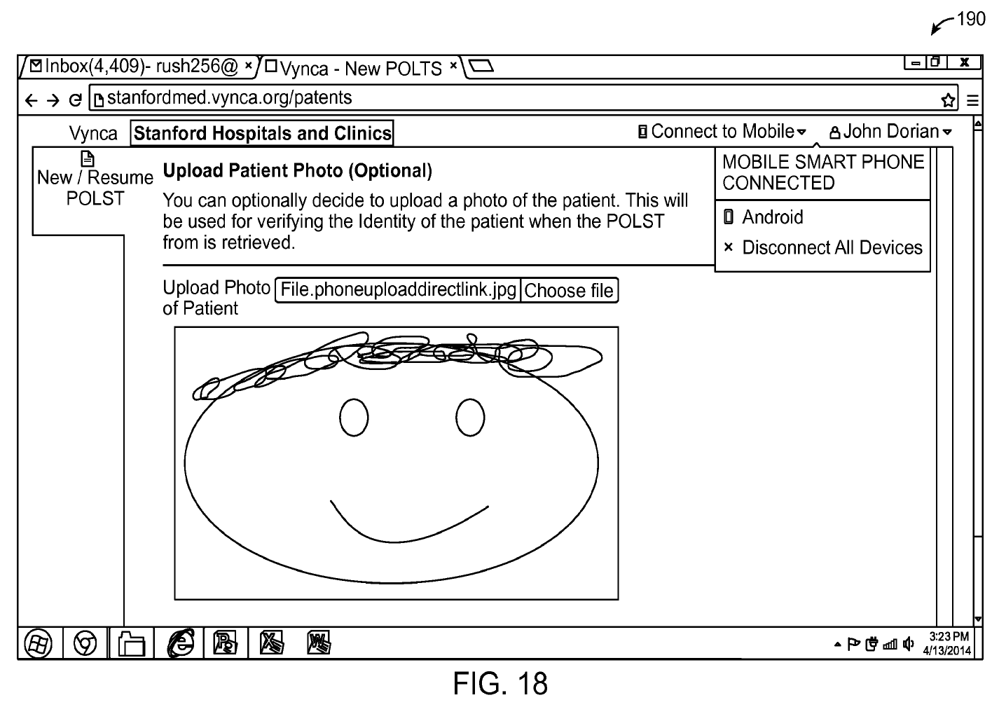

FIG. 18 depicts a screen shot 190 of an exemplary user interface and system for documenting medical information including information useful for patient identification in an emergency, such as a picture, by linking a smartphone to the web session through secure QR code or other link and the display of that information.

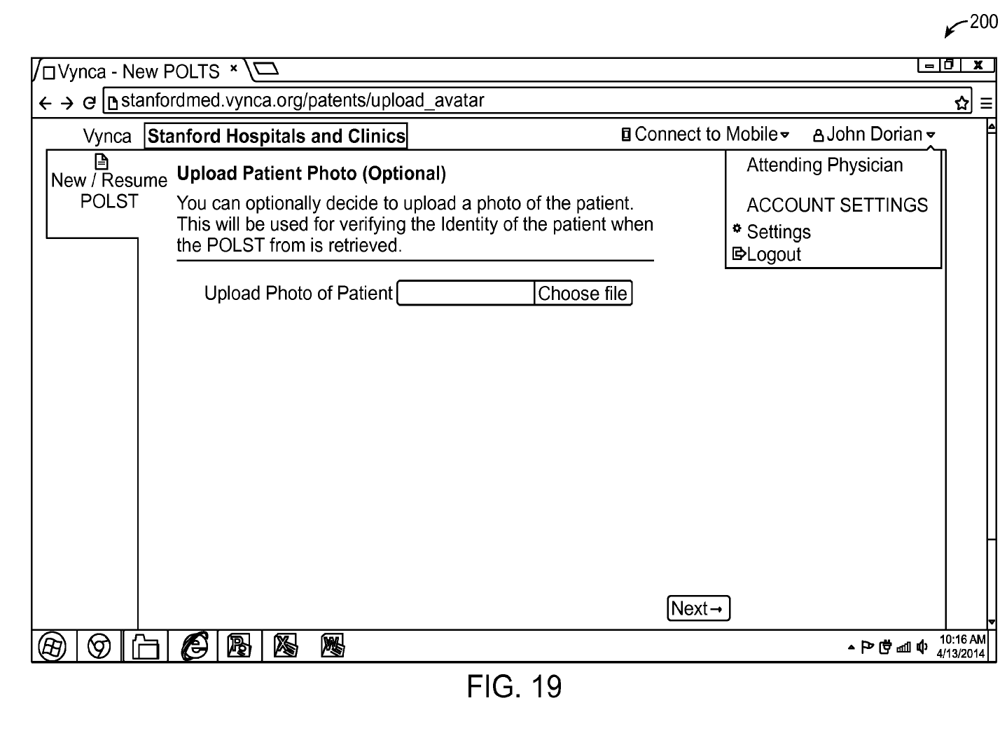

FIG. 19 depicts a screen shot 200 of an exemplary user interface and system for documenting medical information through a secure access portal, including providing a session log-out and settings management tool for the securely authenticated user.

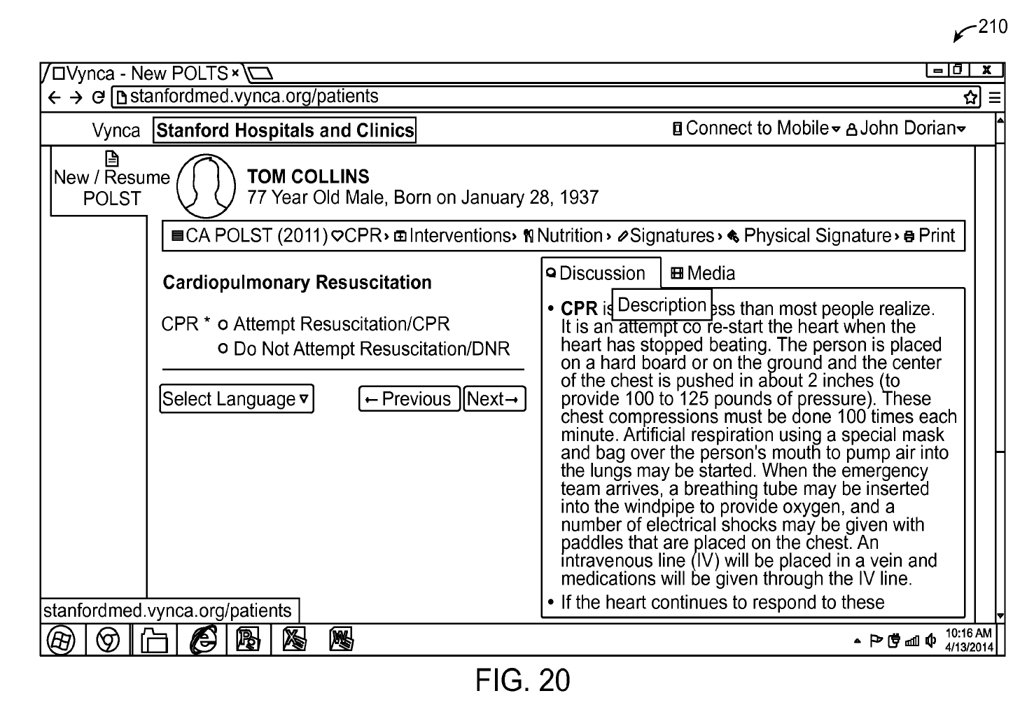

FIG. 20 depicts a screen shot 210 of an exemplary user interface and system for documenting sensitive information, such as a patient's choices on a POLST form, including cardiopulmonary resuscitation, while also providing detailed legal information on such a choice.

FIG. 21 depicts a screen shot 220 of an exemplary user interface and system for documenting sensitive information, such as a patient's choices on a POLST form, including cardiopulmonary resuscitation, while also providing just in time educational information such as videos on such a choice.

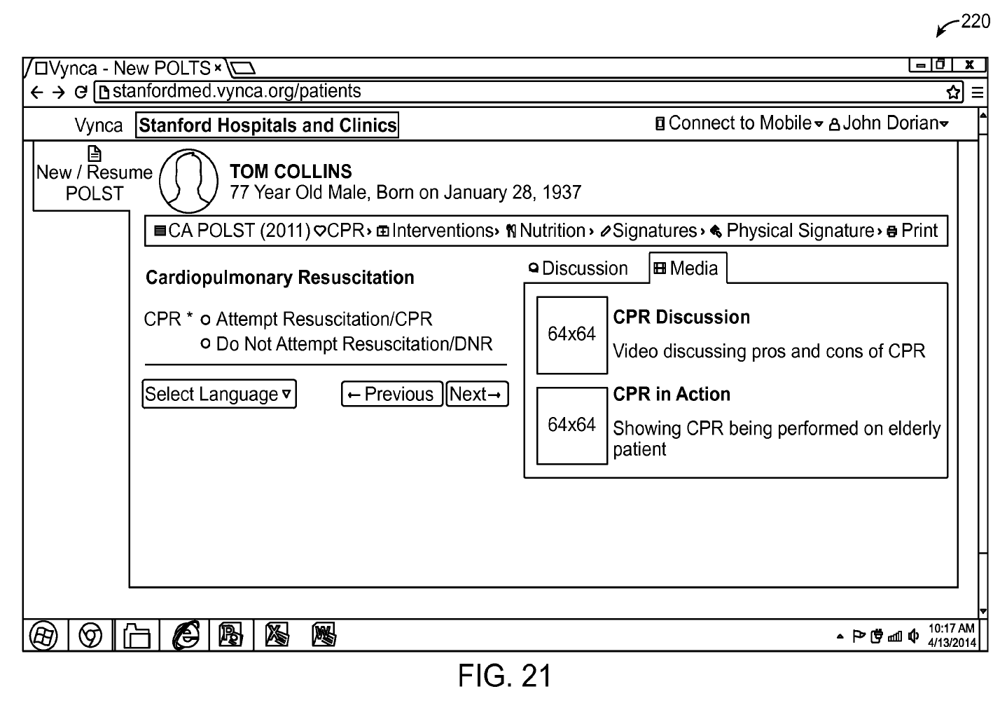

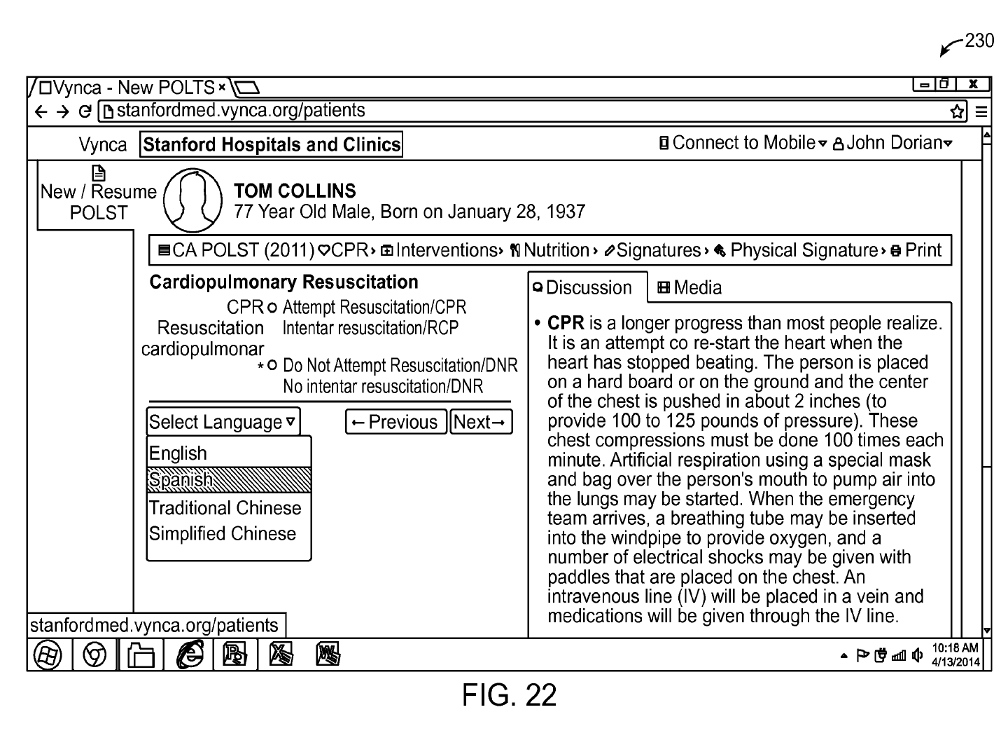

FIG. 22 depicts a screen shot 230 of an exemplary user interface and system for documenting sensitive medical information, such as a patient's choices on a POLST form, and a translation feature that allows for multiple languages to be displayed simultaneously while completing these choices.

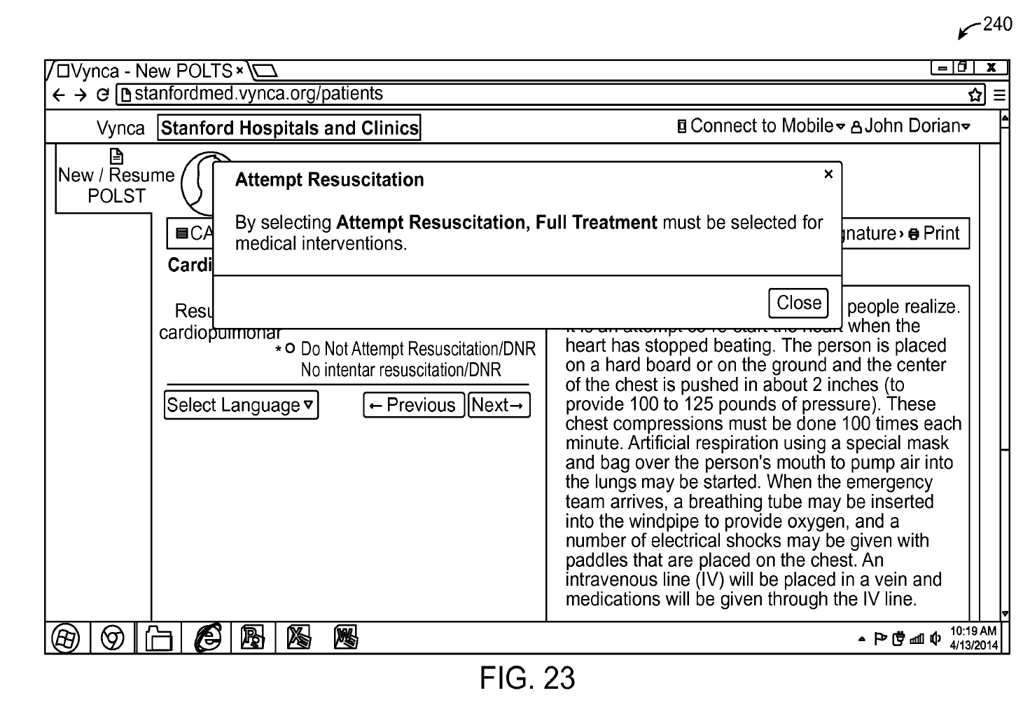

FIG. 23 depicts a screen shot 240 of an exemplary user interface and system for documenting sensitive medical information, such as a patient's choices on a POLST form, and a security and error checking feature that forces compliance with accepted legal guidance around mutually exclusive choices, such as requiring somebody to attempt CPR also defaults to full treatment in a secondary section in some states' documentation.

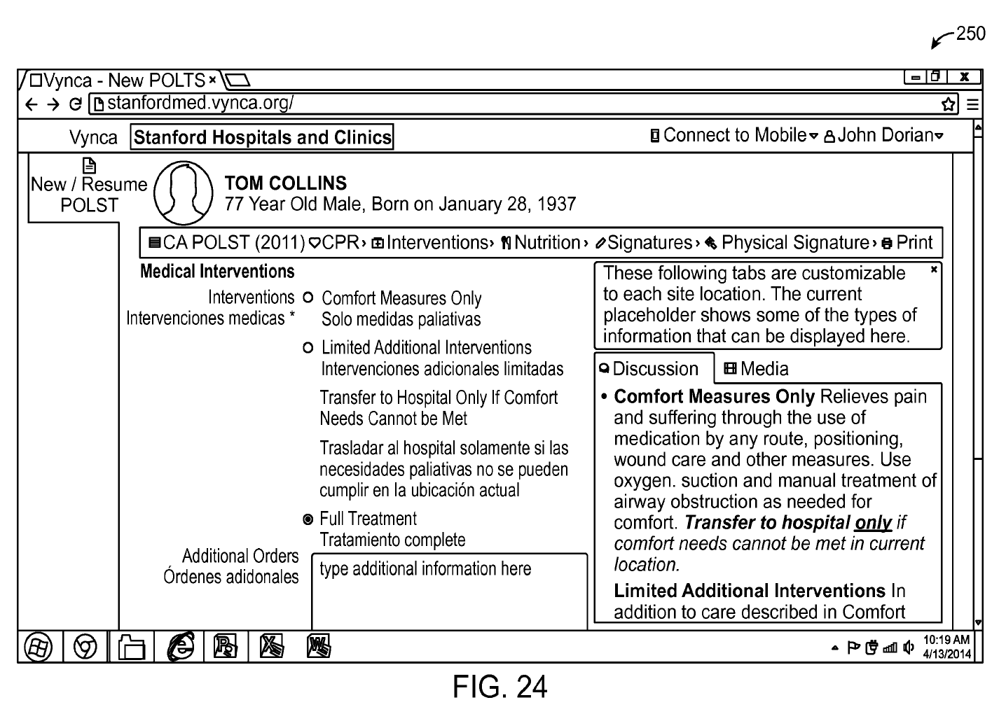

FIG. 24 depicts a screen shot 250 of an exemplary user interface and system for documenting sensitive medical information, such as a patient's choices on a POLST form, and a feature to customize the just in time medical education by various sites.

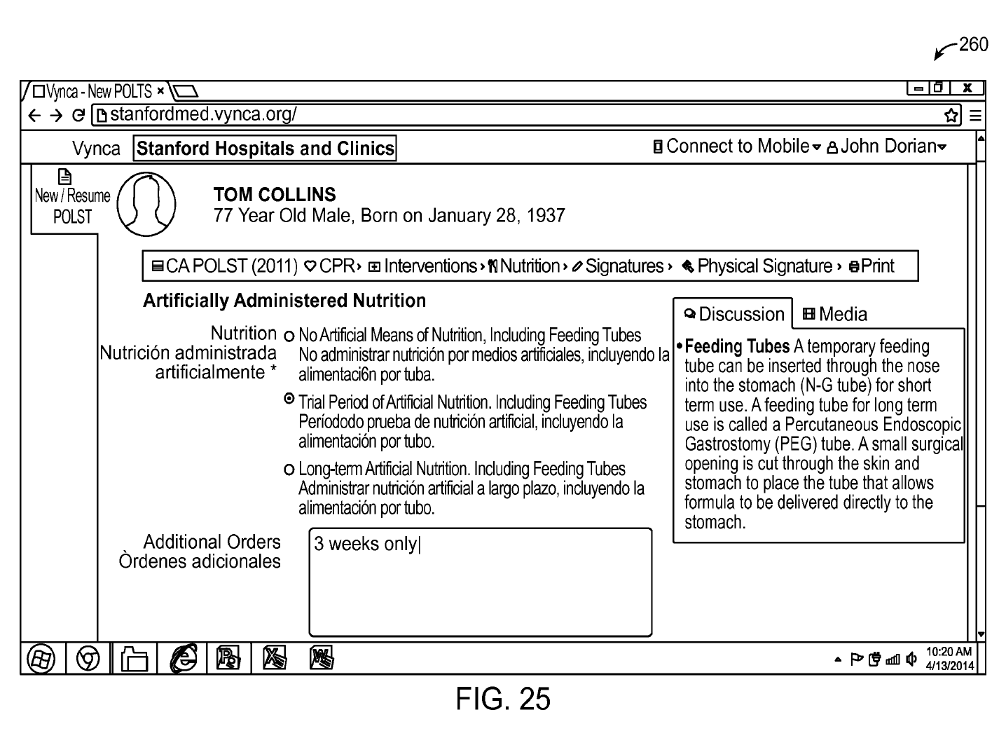

FIG. 25 depicts a screen shot 260 of an exemplary user interface and system for documenting sensitive medical information, such as a patient's choices on a POLST form, for artificial nutrition, including but not limited to entry of specific lengths of time desired, while also providing just in time education and/or legal information on the effects of such a choice.

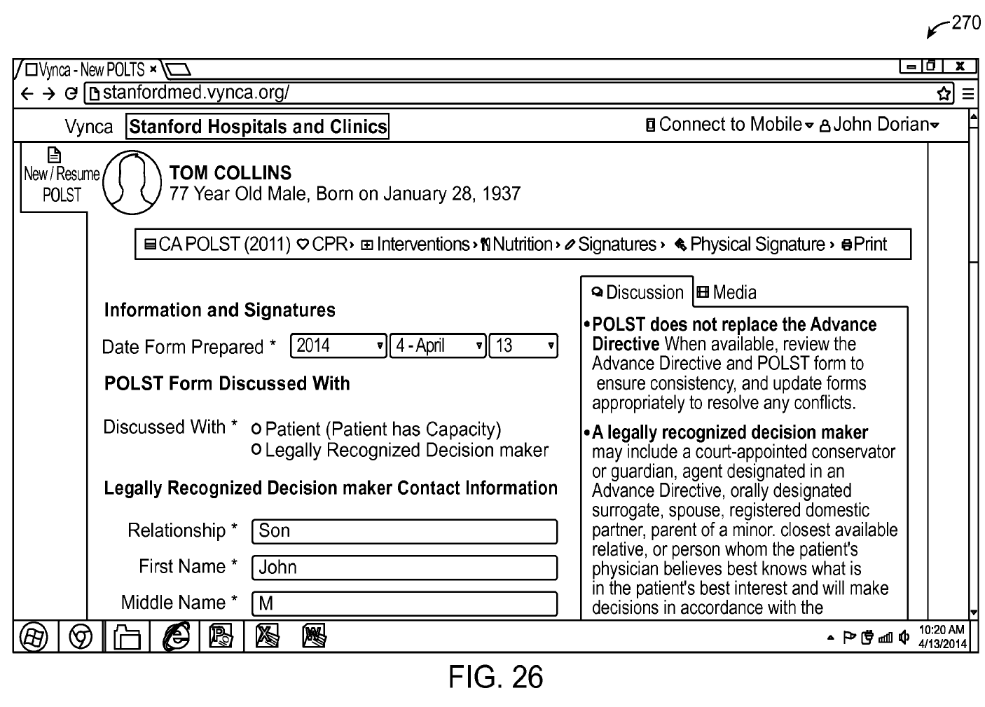

FIG. 26 depicts a screen shot 270 of an exemplary user interface and system for entering demographic information to accompany the process of authenticating and/or signing a document digitally with a signature pad, touch screen, linked smartphone or tablet, and/or electronic documentation means.

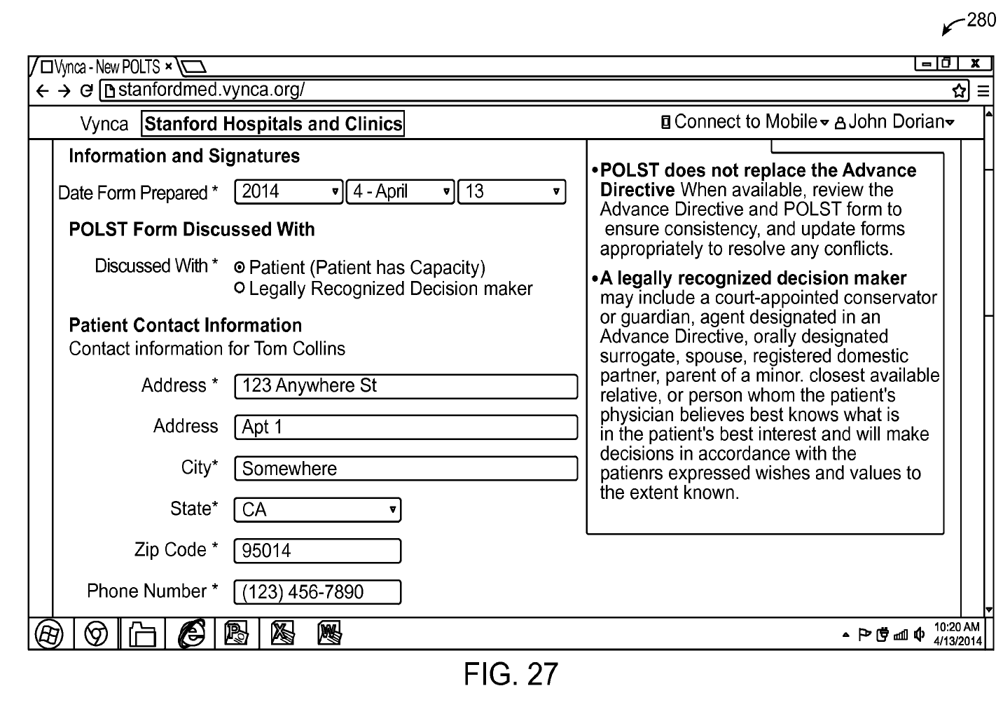

FIG. 27 depicts a screen shot 280 of an exemplary user interface and system for entering demographic information to accompany the process of authenticating and/or signing a document digitally, including auto-populating and/or collapsing entry boxes if, within the sequence of choices, demographic information already entered or auto-populated within another section of the form can be auto-filled or completed into this section.

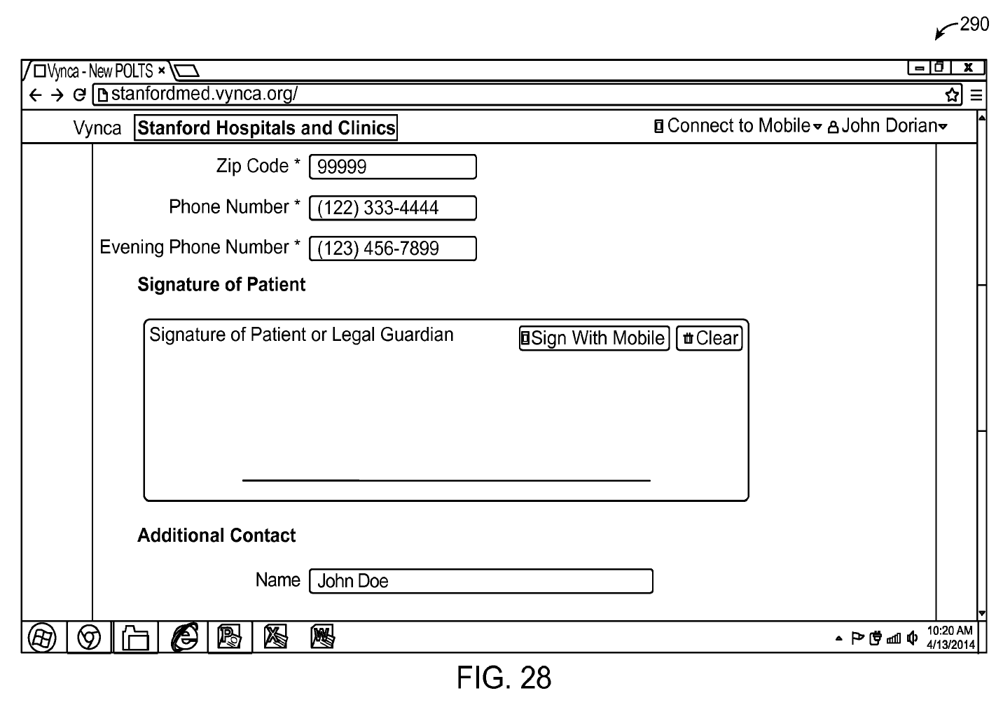

FIG. 28 depicts a screen shot 290 of an exemplary user interface and system for electronically signing an authenticated document.

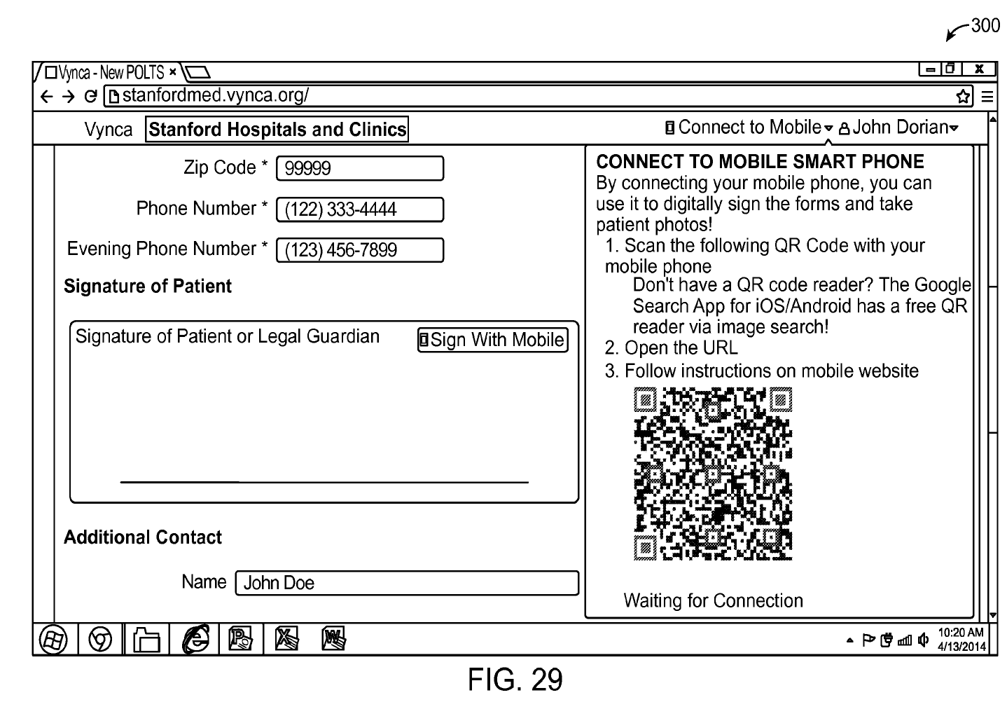

FIG. 29 depicts a screen shot 300 of an exemplary user interface and system for electronically signing an authenticated document by linking a mobile device, such as a smartphone or tablet, via QR code, to a web session to allow for data input from that mobile device to facilitate the unique authentication and signature acquisition.

FIG. 30 depicts a screen shot 310 of an exemplary user interface and system for electronically signing an authenticated document by linking a mobile device such as a smartphone or tablet to a web session while removing the identifying feature such as a QR code once a link has been detected and also providing feedback to the user on the home device as to the type of other device linked to the session to provide guidance to the user of the specific type of device linked.

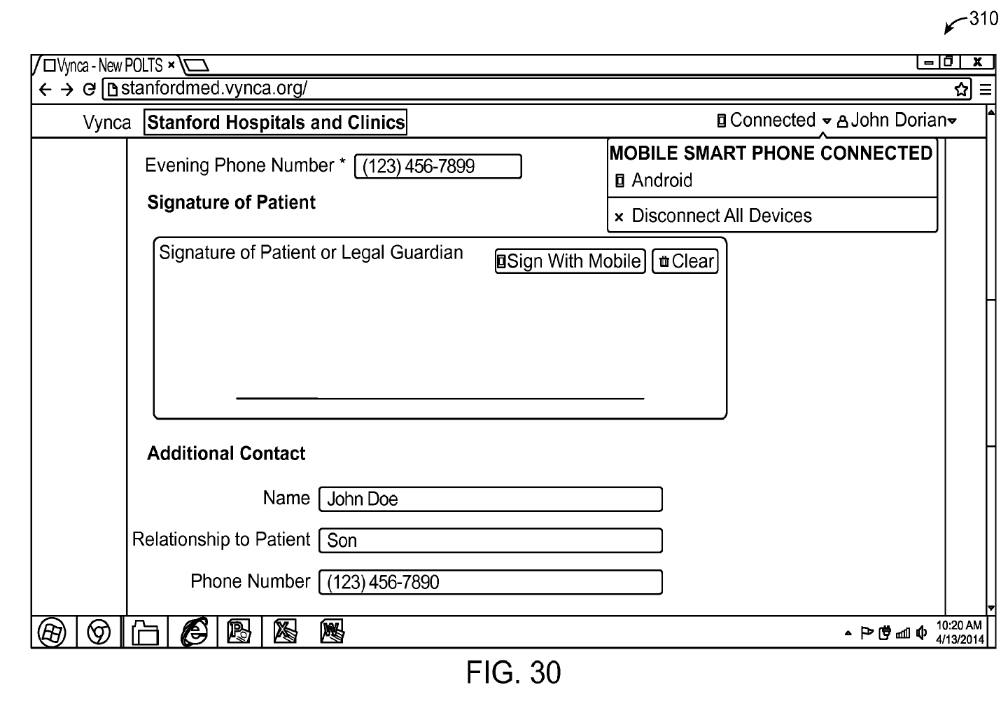

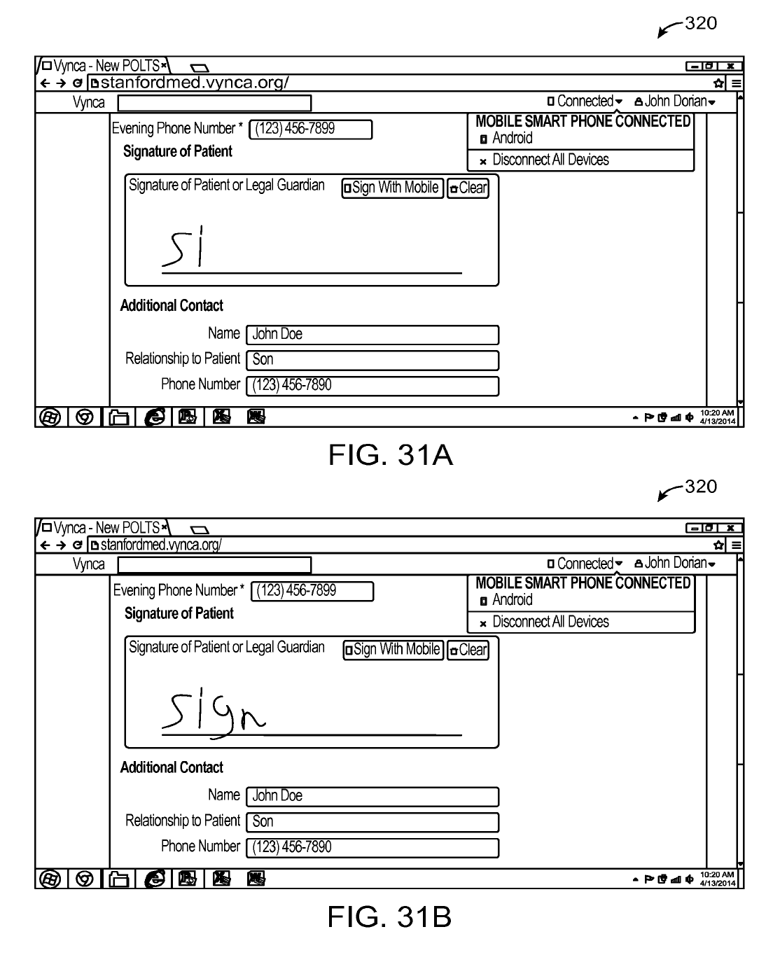

FIGS. 31A and 31B depict screen shots 320 of an exemplary web user interface and system for capturing an electronic signature through a secondary device that was linked to a web session, such as a signature pad or touch screen device, including but not limited to a tablet or smartphone, such that portions of the signature appear in near real time on a home device or home session as soon as they are entered on the secondary device, discounting network lag.

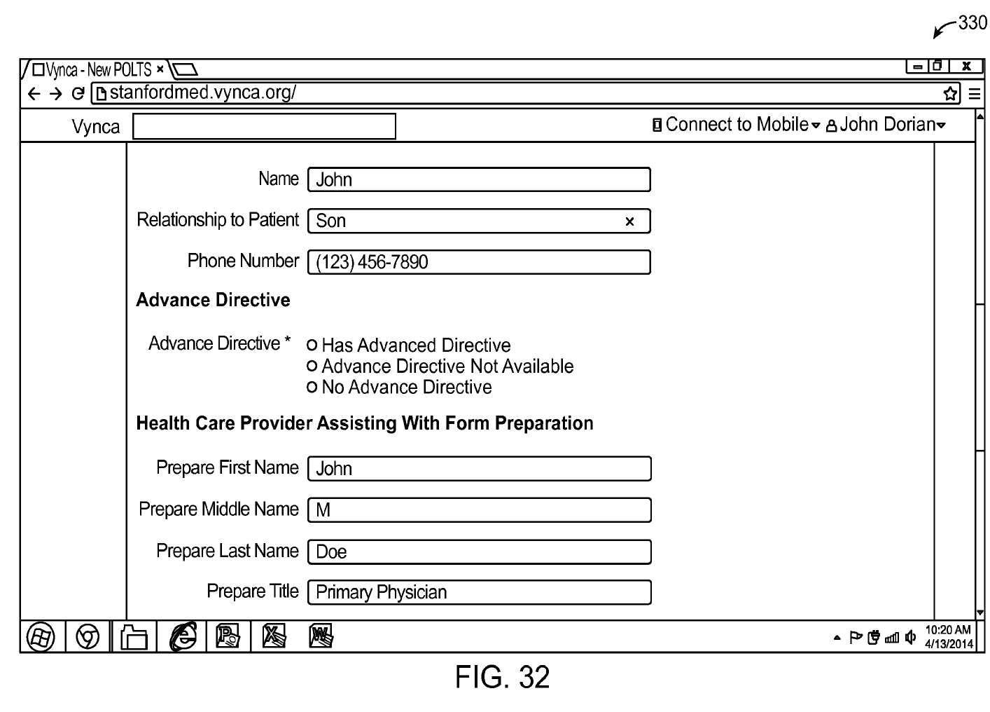

FIG. 32 depicts a screen shot 330 of an exemplary web user interface and system for automatic error checking that all relevant information has been completed when documenting advanced care planning or other type of medical form prior to advancement to another page in the session.

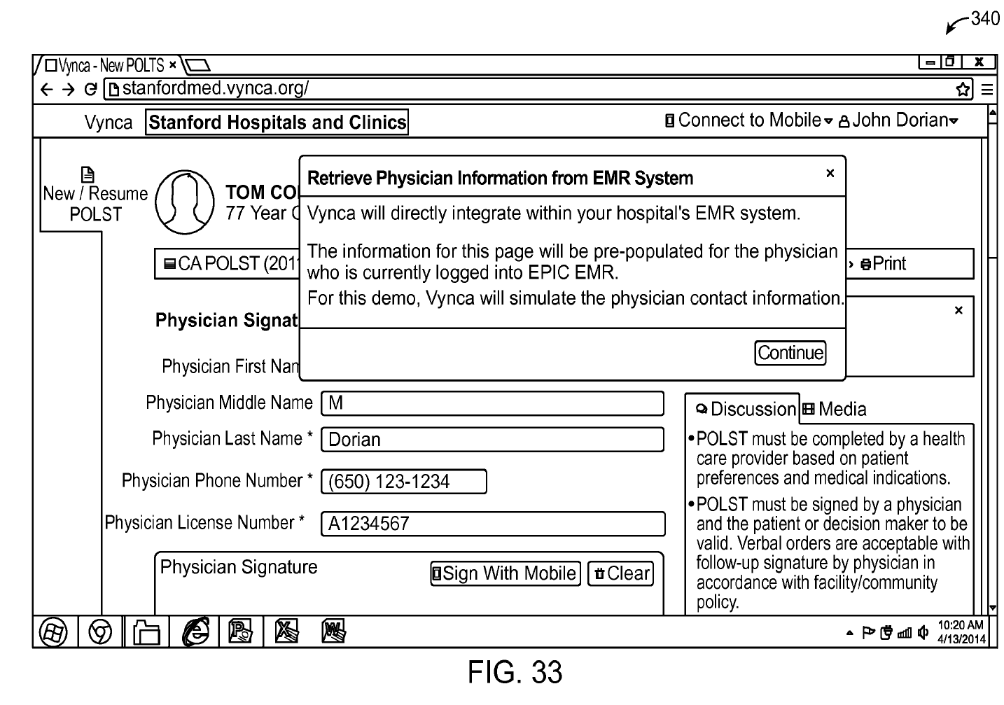

FIG. 33 depicts a screen shot 340 of an exemplary web user interface and system for linking with a medical record and automatically pulling physician and/or healthcare worker information from that medical record to auto-populate the physician and/or healthcare worker information onto a medical form and an acknowledgement to the process of automatic population of that information to the user.

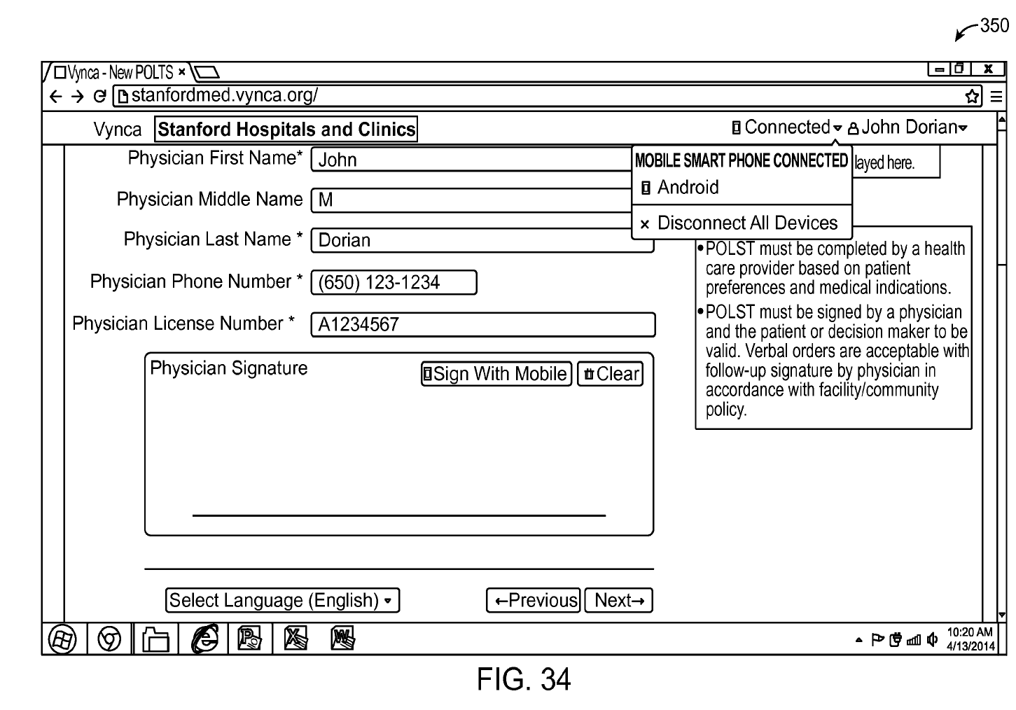

FIG. 34 depicts a screen shot 350 of an exemplary web user interface and system for linking with a medical record and providing signatures for medical documentation through secondary device linking, such that once the secondary device is linked once it is allowed to change by directed guidance from a home computer or system, which it was linked to, in order to allow for multiple signatures to be entered in sequence without requiring separate device linking for each signature capture.

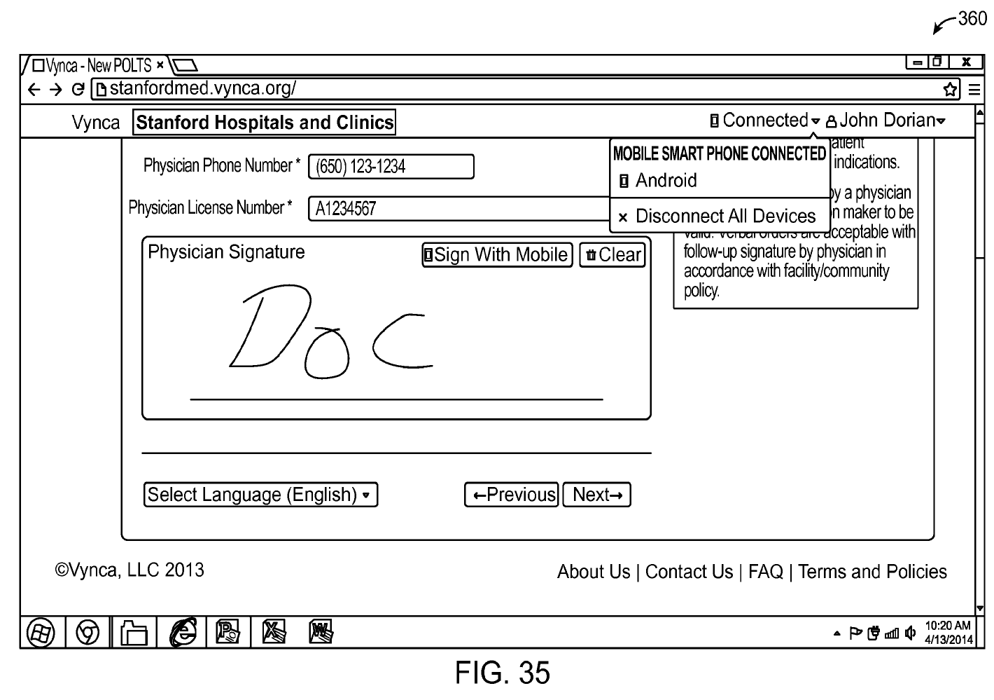

FIG. 35 depicts a screen shot 360 of an exemplary user interface and system for capturing an electronic signature image from a secondary device through a web link to a primary device.

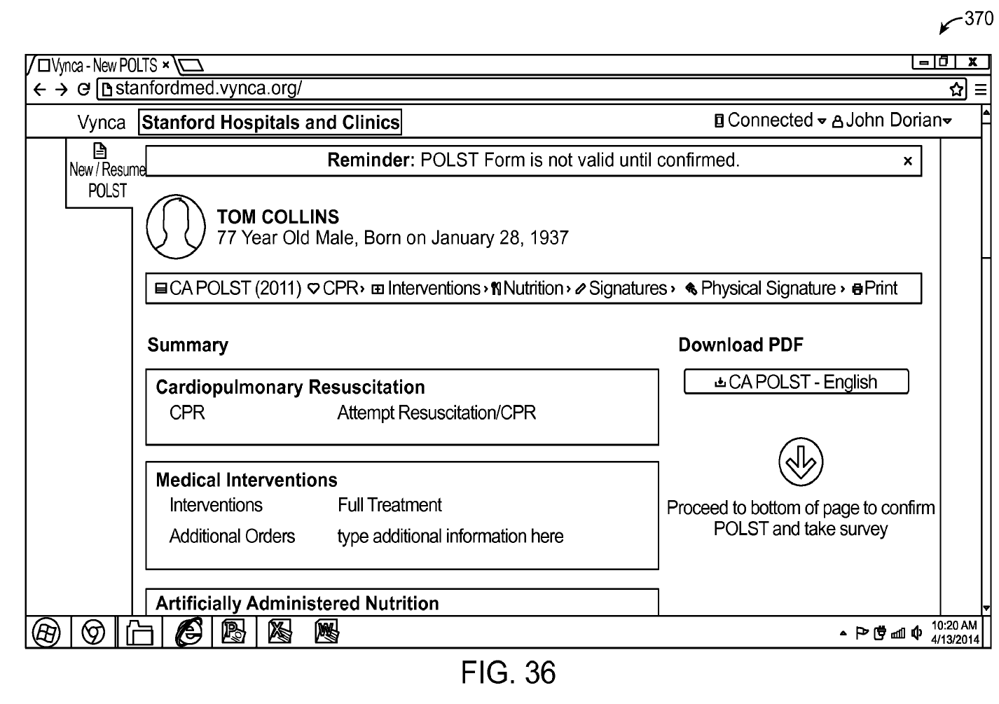

FIG. 36 depicts a screen shot 370 of an exemplary user interface and system for capturing medical information on a web form including a summary page for review of that information with or without a reminder to confirm the validity of the summary before the document is active.

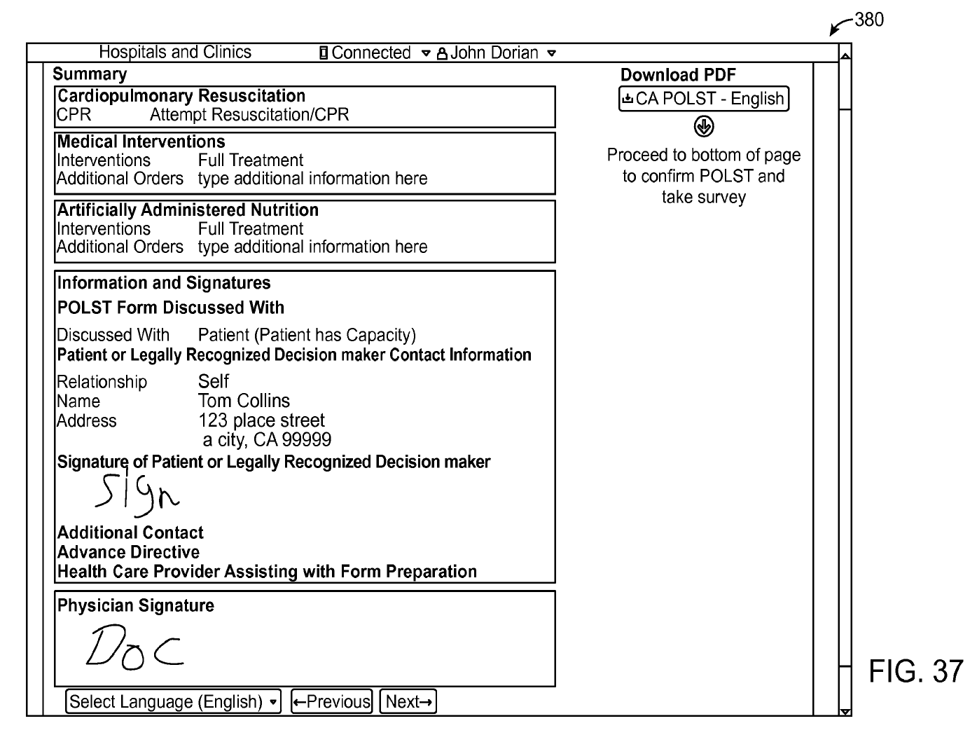

FIG. 37 depicts a screen shot 380 of an exemplary user interface and system for capturing medical information on a web form, including a summary page for review of that information before the document is active.

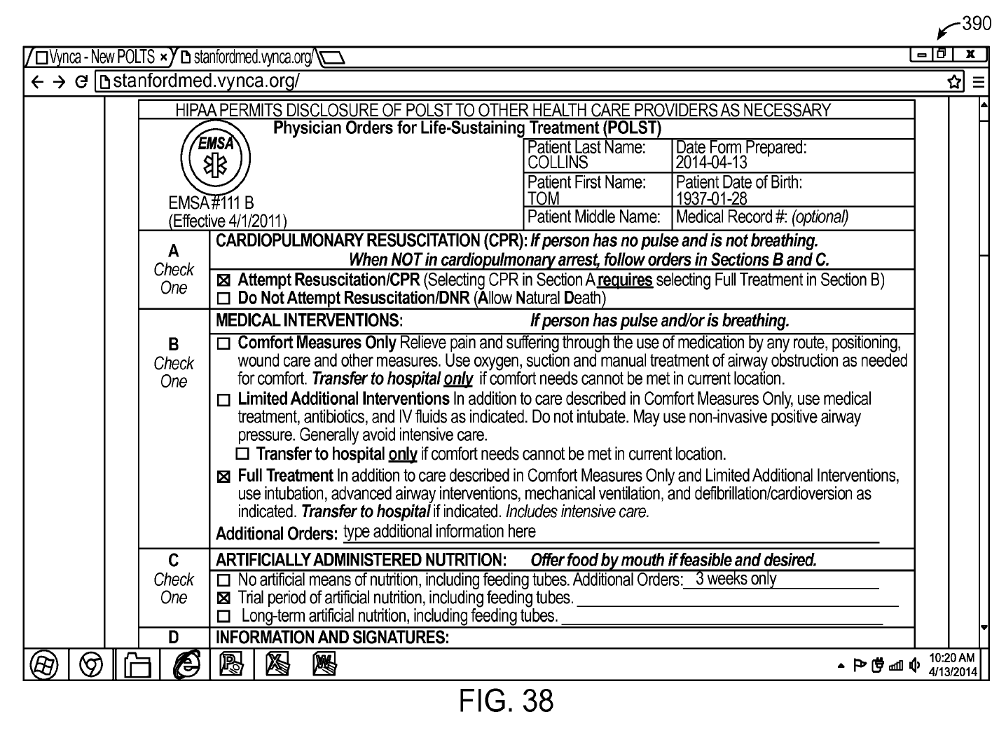

FIG. 38 depicts a screen shot 390 of an exemplary user interface and system for capturing medical information on a web form such a POLST document including converting web form populated information into the appropriate document that can be printed and sent home with a patient as well as stored within an electronic medical record, stored in a cloud, or accessed remotely through a medical record or other secure authenticated access portal.

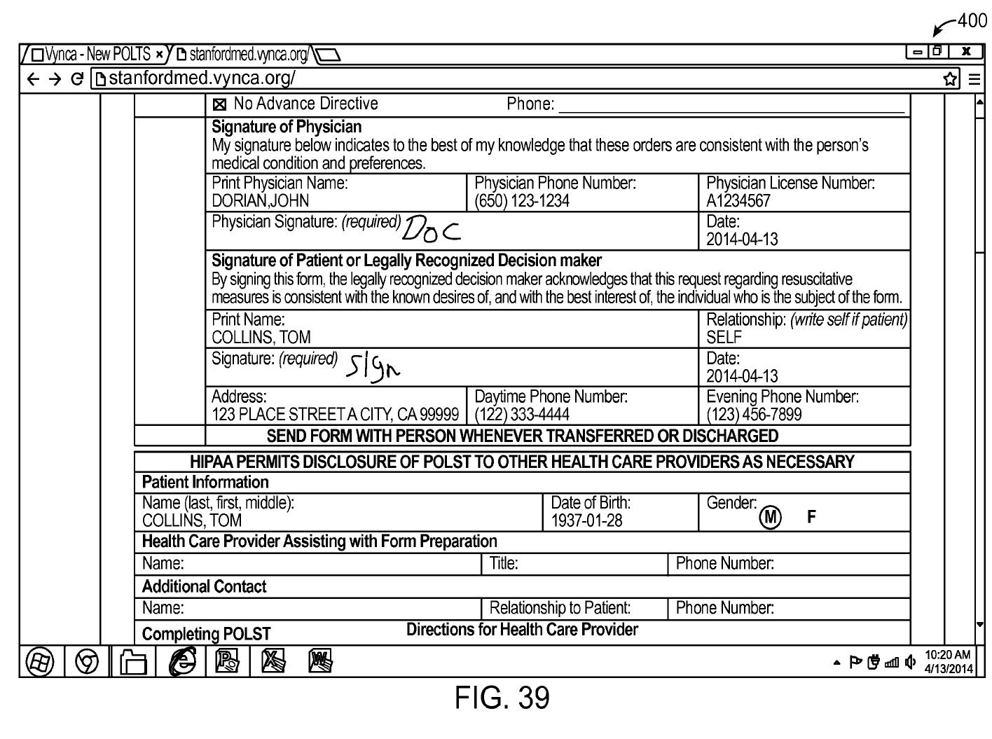

FIG. 39 depicts a screen shot 400 of an exemplary user interface and system for capturing medical information on a web form such a POLST document including converting web form populated information into the appropriate document that can be printed and sent home with a patient including time and date stamp information for when the form was completed and signed.

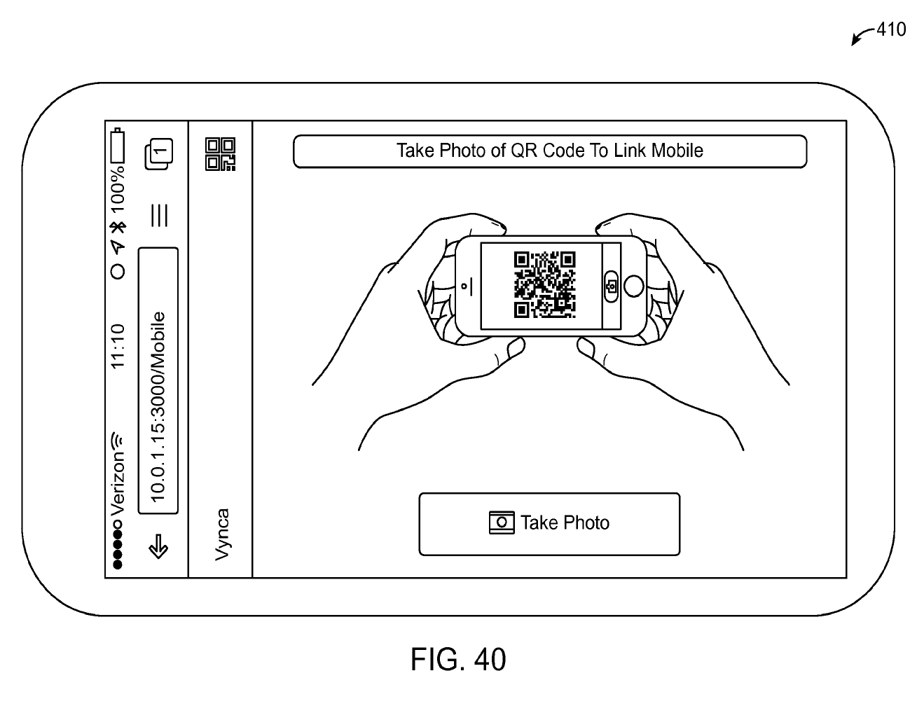

FIG. 40 depicts a smart phone 410 for use with an exemplary user interface and system for capturing an electronic signature image for a medical form from a secondary device through a web link to a primary device by taking a photo through a web based QR code reader.

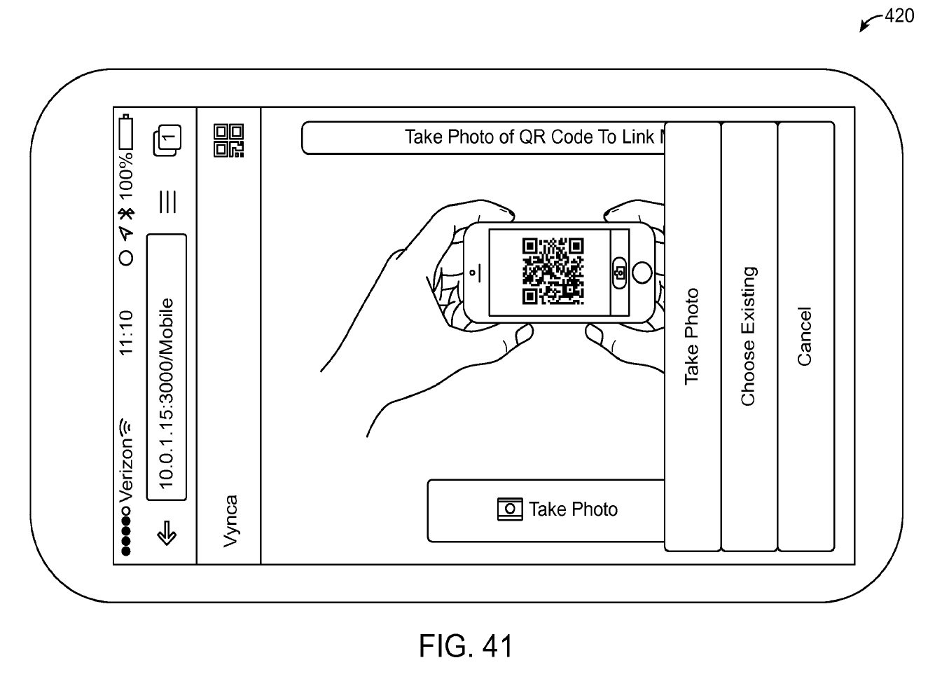

FIG. 41 depicts a smart phone 420 for use with an exemplary user interface and system for capturing an electronic signature image for a medical form from a secondary device through a web link to a primary device by taking a photo through a web based QR code reader that activates the camera on the phone and directs a user to take a photo while also displaying appropriate educational information on correct orientation.

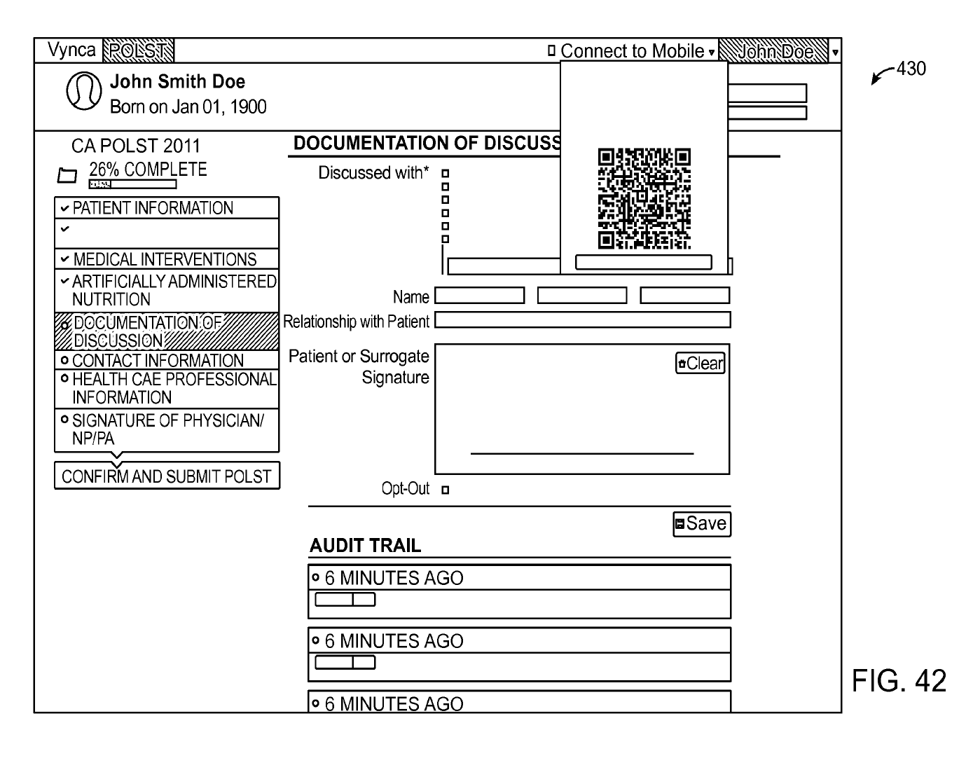

FIG. 42 depicts a screen shot 430 of an exemplary user interface and system for completing a medical form via a web interface that is behind an authenticated wall such as single sign on from a medical record system or other secure portal such that a QR code or other linking means is displayed after clicking a “connect to mobile” and/or “sign with mobile” link or button such that this provides a secure way to link a secondary device to the web session for data capture including a signature image.

FIGS. 40-51 illustrate one embodiment for linking a mobile phone through a website, web app, or downloaded app to the system's website, to have different levels of functionality. One important feature is the capability not only to push data from the phone to the EHR computer, but also to provide pushed data that includes a patient education or functional side-by-side interface experience. Also, the system may display a linking icon or other active indicator, once the phone is linked to the system.

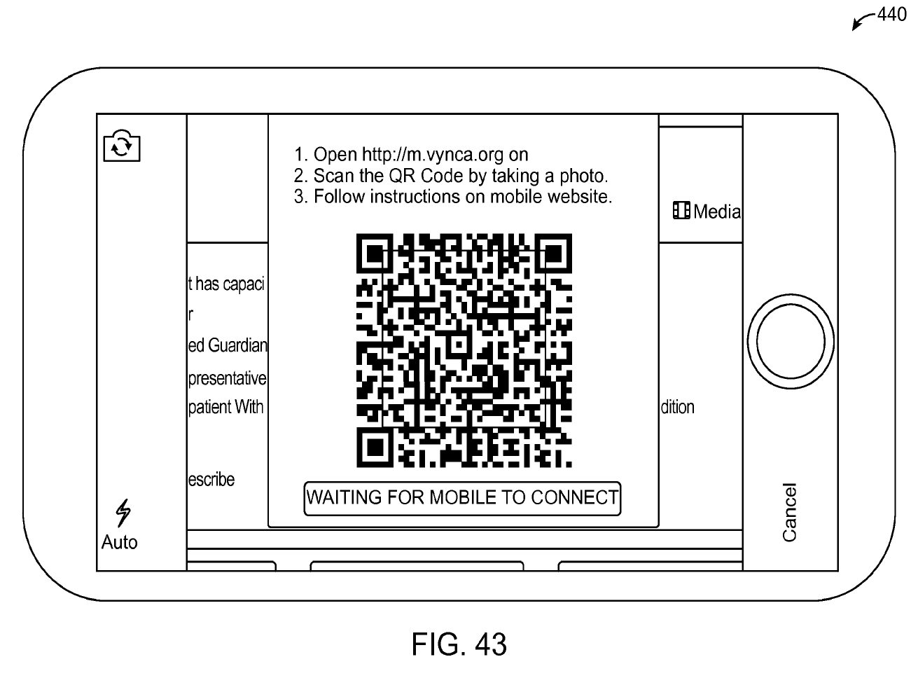

FIG. 43 depicts a smart phone 440 for use with an exemplary user interface and system for linking the smart phone 440 with a secure web interface by taking a picture of a QR code through a website or scanning a QR code via an installed app or web app to link the phone 440 to the web session.

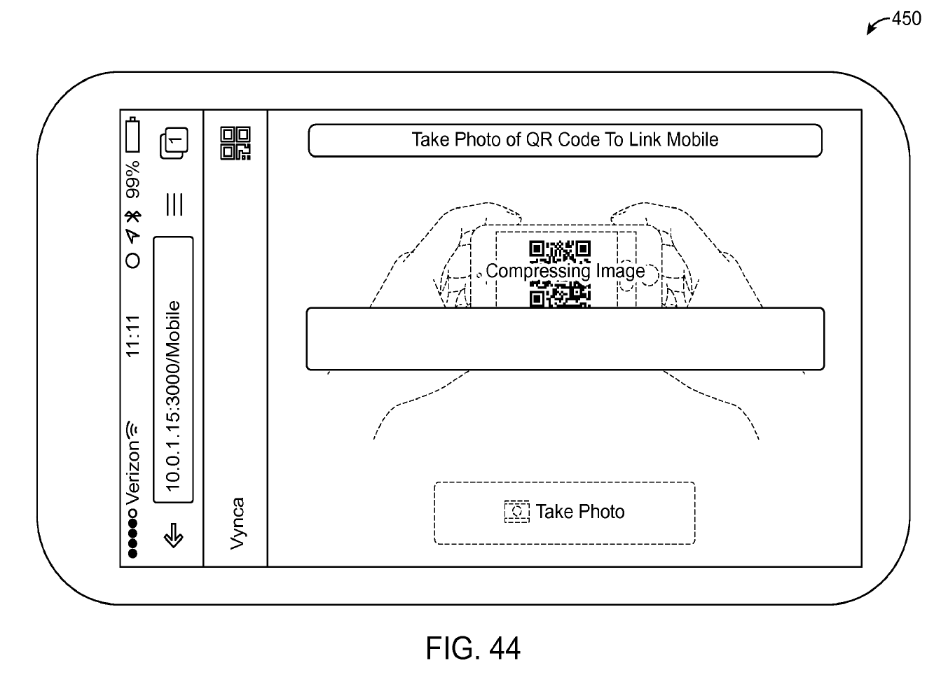

FIG. 44 depicts a smart phone 450 for use with an exemplary user interface and system for linking the smart phone 450 or tablet to a web interface for completing forms including medical forms such as POLST while alerting the user as to time to link the devices by showing a compressing image and/or a progress bar on the phone 450 once the QR code or other identifying linking feature has been captured and is being processed by the secondary device such as the smart phone 450 or tablet.

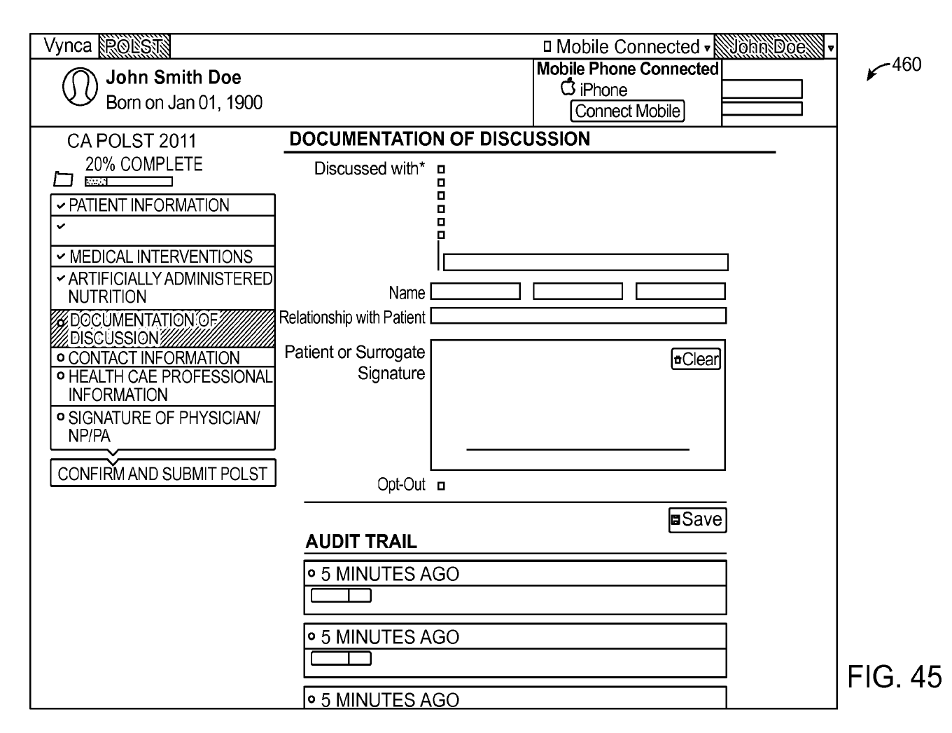

FIG. 45 depicts a screen shot 460 of an exemplary user interface and system for completing a web based form including a POLST form or other medical documentation such that once a secondary data device is linked the web session will display identifying information about that device such as its make, or model, or other feature that can guide the user as to its secureness as well as once linked the web session will remove or hide the identifying mark such as a QR code from being able to be scanned by any other devices.

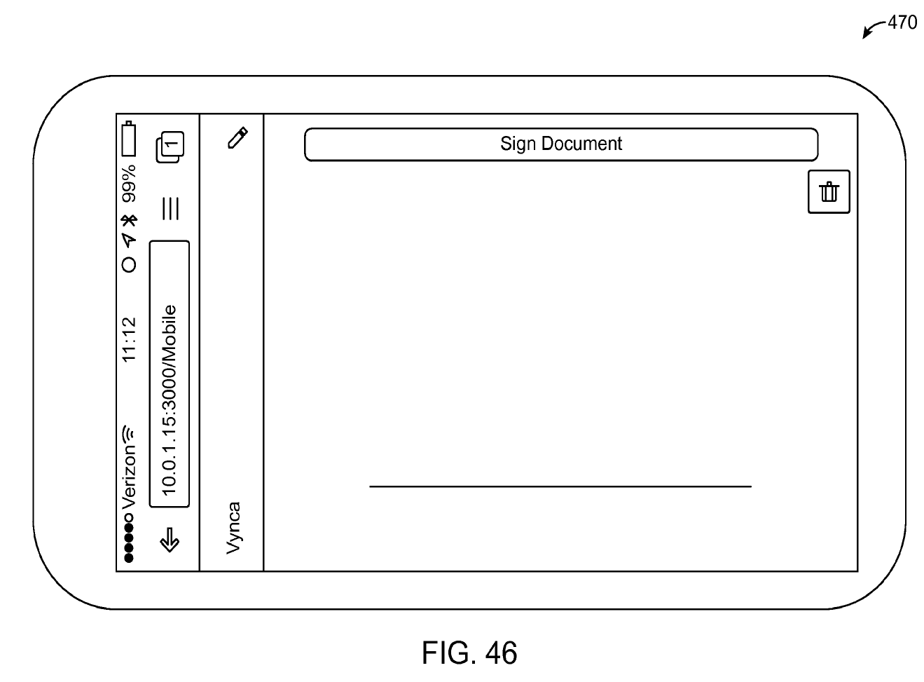

FIG. 46 depicts a smart phone 470 for use with an exemplary user interface displayed on the smart phone 470 or tablet after linking has occurred to the web session. This interface allows a user to sign a document and provides an alert to the feature activated on the linked device, such as “sign document,” “take photo,” “scan fingerprint,” “shake phone,” “press record,” “start speaking,” “click to begin video,” or other phrase or feature that may be desired, depending on what type of data acquisition feature has been activated by directions from the web session to the linked smart phone 470 or tablet device.

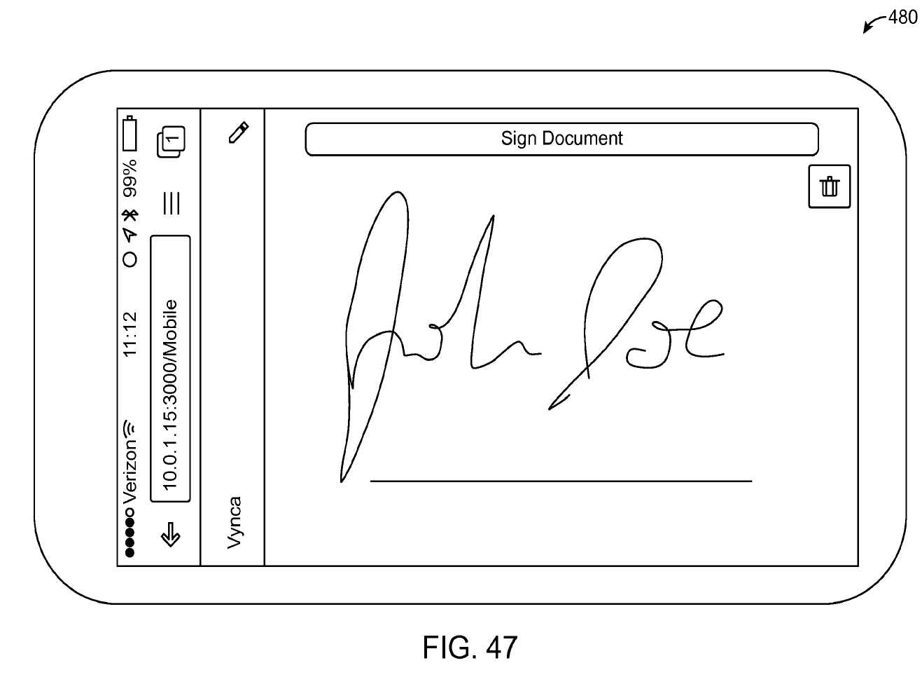

FIG. 47 depicts a smart phone 480 for use with an exemplary user interface and system for capturing and displaying a signature image on a secondary device that is linked to a web session on a primary device that is entered through the touch screen on the secondary device.

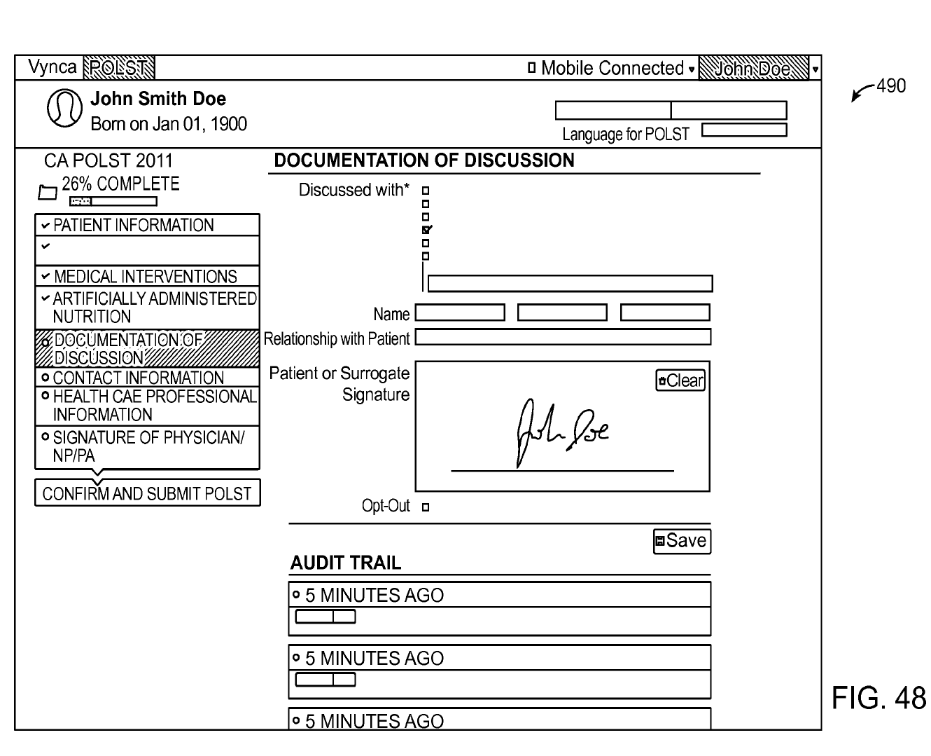

FIG. 48 depicts a screen shot 490 of an exemplary user interface and system for completing a web form, including a POLST or other type of medical and/or advanced care planning form, such that a signature image is displayed in near real time and/or real time depending on lag between linked devices, on a signature page of a web session displayed on a primary or home device while or shortly after the signature is signed on a secondary device with a touch screen such as a tablet or smartphone.

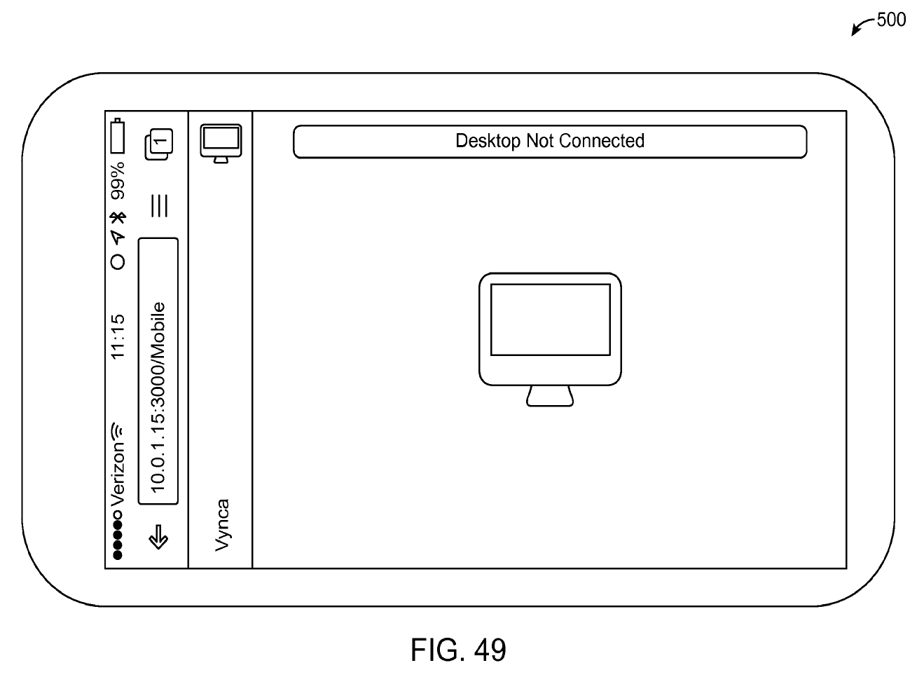

FIG. 49 depicts a smart phone 500 for use with an exemplary user interface and system for linking a mobile device such as a tablet or smartphone to another device via a web session including an indicator feature that a session has timed out, been disconnected, or is no longer linked to the home device through the web session.

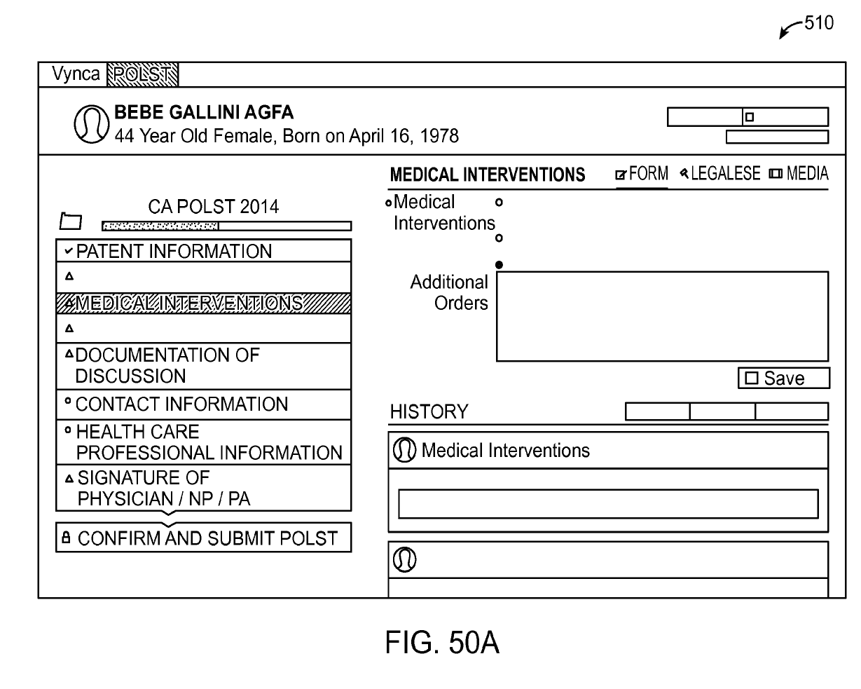

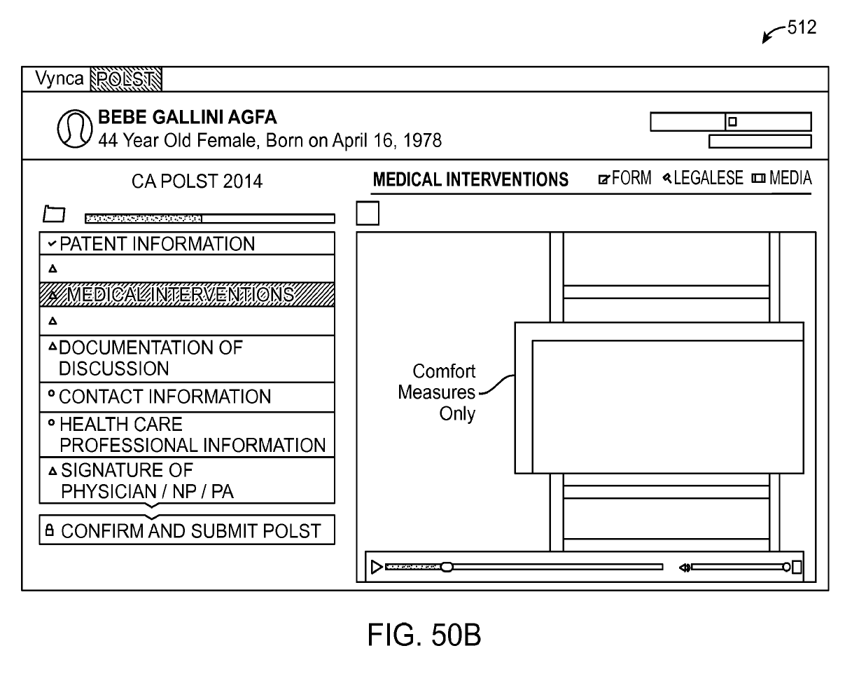

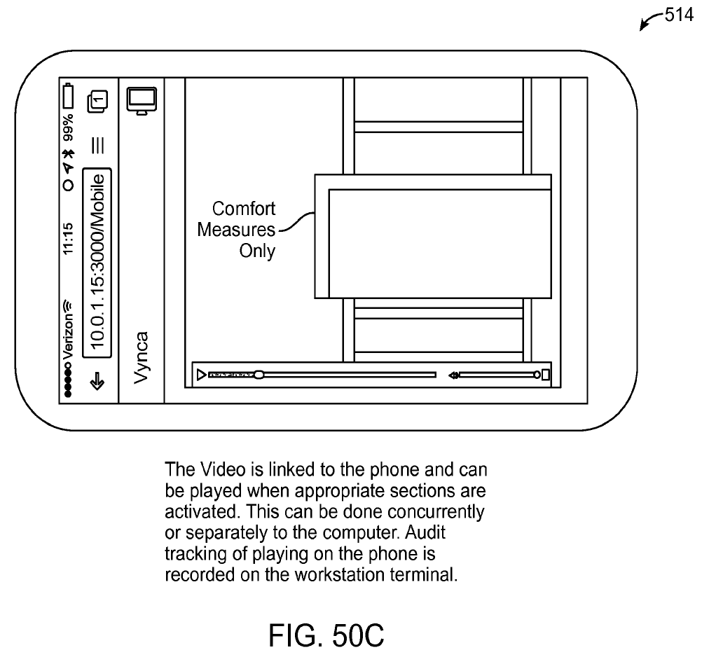

FIGS. 50A-50C depict a screen shot 510 (FIG. 50A), another screen shot 512 (FIG. 50B) and a smart phone 514 (FIG. 50C) that are part of or used with an exemplary user interface and system for linking a mobile device, such as a tablet or smart phone 514 to another device via a web session, including passing information from the other device to the tablet or smart phone 514, such as a video to be played on the tablet or smart phone 514. This includes the phone 514 following the activity of which section is currently active on the computer terminal such that a specific page must be opened in order to allow for the specific smart phone feature for that page to be active.

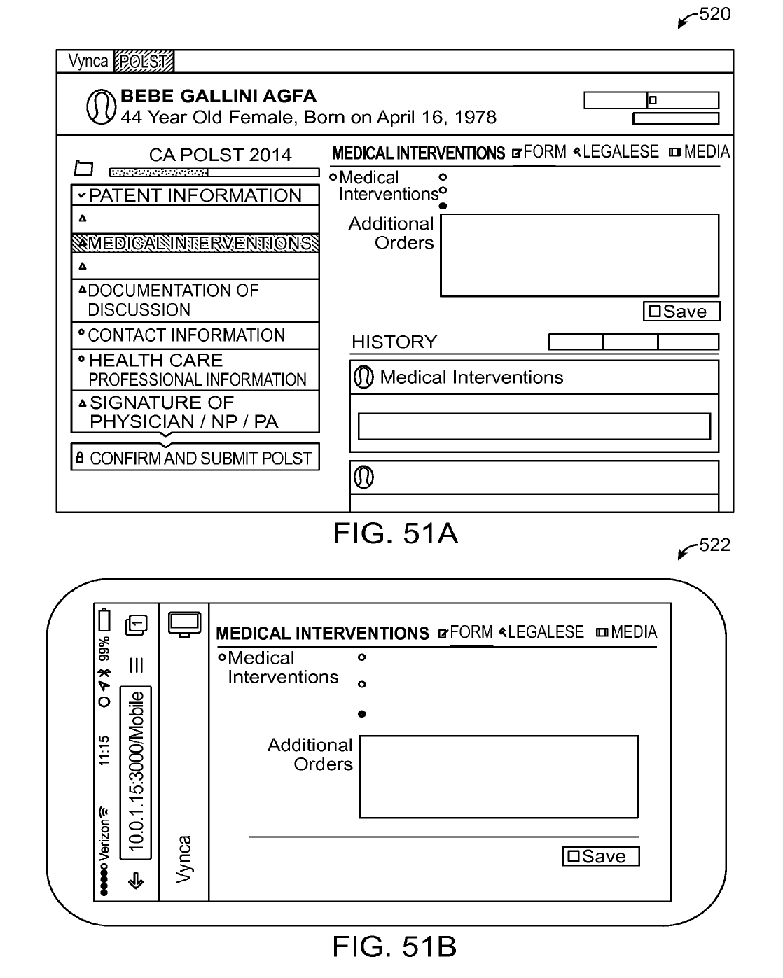

FIGS. 51A and 51B depict, respectively, a screen shot 520 and a smart phone 522, which are part of or for use with an exemplary user interface and system for linking a mobile device such as a tablet or smart phone 522 to another device via a web session including passing information from the another device to the tablet or smart phone 522, such as a form interface system, without identifying information that would cause information entered on the smartphone or tablet linked device to be constituted as identified HIPAA information, but instead the identifiers and the information entered on the smart phone 522 or tablet linked devices is otherwise sent together to the system and stored securely and may be accessed in its entirety on the secure another device such that information can be entered and then sent to the system which may also securely record the actions taken on the linked devices to create an audit tracking event of activity recorded in the secure system on the activities of the unsecure interface on the tablet or smart phone 522.

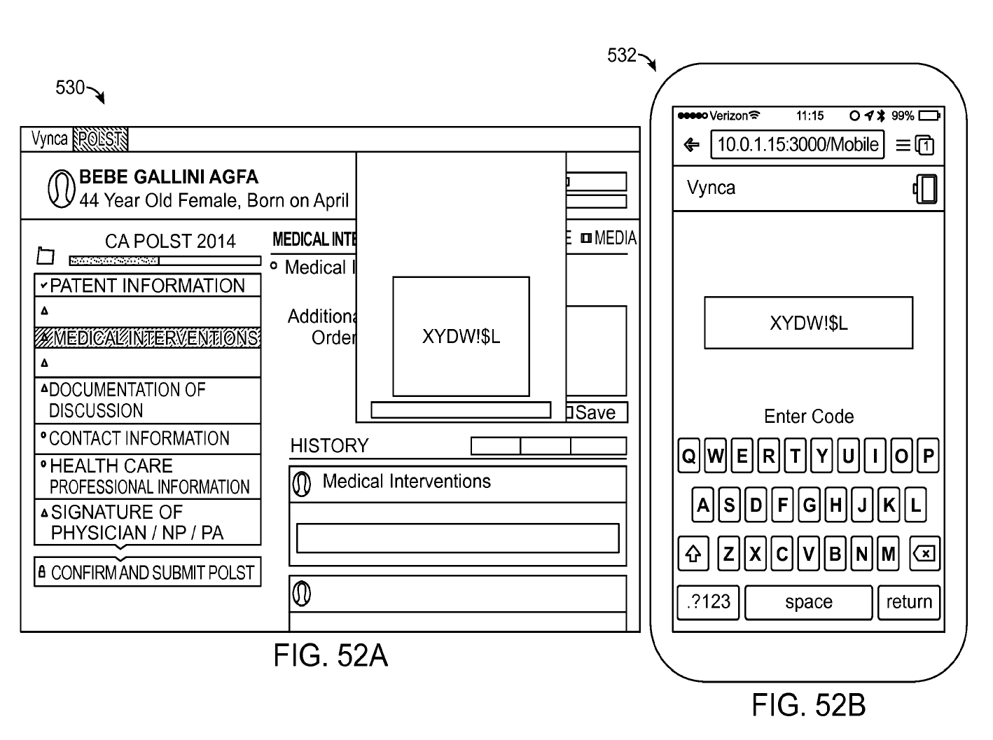

FIGS. 52A and 52B depict, respectively, a screen shot 530 and a smart phone 532, which are part of or for use with an exemplary user interface and system for linking a mobile device such as a tablet or smart phone 532 to another device via a web session by clicking a section of the another device user interface to display a linking code which is temporary in that the linking code would be entered on a web page or app window opened on the tablet or smartphone to link to the specific web session.

Using the system illustrated in FIGS. 52A and 52B, a user may log into the EHR, click a link to access the system (labeled “Vynca” in the figures), and then click the link to connect to the mobile device 532. The system may then display a code of letters, code of colored boxes, or some other mechanism, so that the mobile device 532 may have entry features on it to link the mobile device 532 and the computer. In some embodiments, the mobile device 532 may go to a website and display an open entry feature, and the user may then be required to enter a correct code. In some embodiments, certain codes are only activated after the user has gone through several steps on the PC side, to provide a secure mechanism to link the mobile device 532. In this way, the system may link the IP address to a geographic region and/or the signal, GPS location, IP address or the like of the mobile device 532 to a similar region to narrow down the number of variables even further and produce a higher likelihood of a hit to link the mobile device 532.

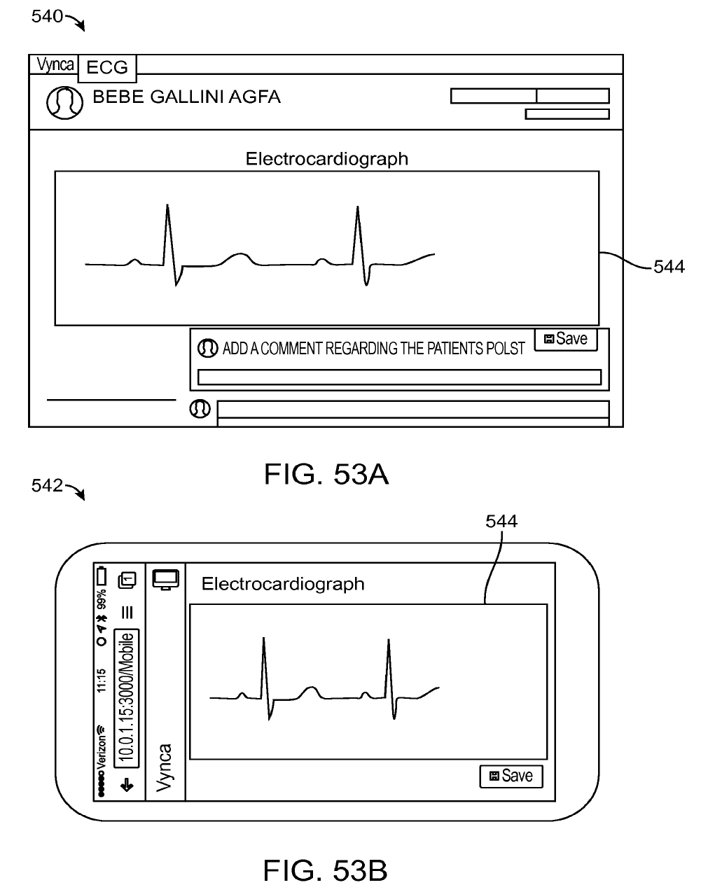

FIGS. 53A and 53B depict, respectively, a screen shot 540 and a smart phone 542, which are part of (or for use with) an exemplary user interface and system for linking a mobile device, such as a tablet or smart phone 542, to another device via a web session. The screen shot 540 and the smart phone 542 are simultaneously displaying an electrocardiograph 544. Linking may be achieved using a QR code or other linking system to link the smart phone 542 to a user's account session and/or prompting the instillation of a security certificate, cookie, or other identifying token or password to allow the mobile device to automatically link to the system running on the another device when the another device terminal has been logged into, such that a user can then leave the area with the smart phone 542 actively transmitting data to the medical record, such as when the electrocardiogram is activated.

FIGS. 53A and 53B illustrate that the system may, in some embodiments, link the smart phone 542 and have various other types of input data, such as an electrocardiograph 544 or any other type of data that could be entered into a mobile device through an add-on device to that mobile device or to a sensor that is embedded within that mobile device itself. In alternative embodiments, data entered from the smart phone 542 to the electronic medical record may be displayed on the PC and then once the smart phone 542 is linked, the displayed data could be pushed to the smart phone 542 or tablet, so that it could be viewed by the patient in their bed, wheelchair or on their person while they are walking around, traveling, etc., so that they do not need to be in the same direct vicinity of the computer to view the computer screen.

While many of the exemplary embodiments involve healthcare data and health information, it should be understood that the invention may be applied to other domains and uses, including but not limited to consumer-facing web sites, financial services, general corporate or business applications or any other application where there is a desire to link the data acquisition and data display functionality of two devices and their associated web sessions, application or other software.

What is claimed is:

1. A method for providing access to patient information from within an electronic medical record, the method comprising:
providing a link on an electronic medical record of a patient, wherein the link is preloaded with a URL parameter;
encrypting the URL parameter;
decrypting the URL parameter responsive to the link being accessed by a user, without requiring the user to provide any further information;
searching a database for provider information and patient information using the decrypted URL parameter; and
providing data associated with the provider information and the patient information to the user responsive to the provider information and the patient information being found in the database.

2. The method of claim 1, wherein the database is populated, in part, with data from a Health Level 7 feed from a healthcare institution at which the patient received healthcare.

3. The method of claim 2, further comprising locating a Health Level 7 message in the database, wherein the message is from the Health Level 7 feed.

4. The method of claim 3, further comprising:
loading patient demographic or healthcare identifiers from the Health Level 7 message; and
locating one or more records within the database that match the patient demographic or healthcare identifiers.

5. The method of claim 4, further comprising providing the located one or more records on a webpage for access by and interaction from the user.

6. The method of claim 5, wherein the data associated with the provider information and the patient information are provided via a display monitor of a computer, and wherein the method further comprises linking the computer to a mobile electronic device to allow the data associated with the patient information to be viewed on the mobile electronic device and on the display monitor.

7. The method of claim 6, further comprising providing a mobile electronic device connection link on the display monitor of the computer to allow the computer to link with the mobile electronic device.

8. The method of claim 7, further comprising:
displaying a code on the mobile electronic device in response to the user clicking on the mobile electronic device connection link; and
receiving the code entered into the mobile electronic device by a mobile device user, wherein linking the computer to the mobile electronic device is not performed until the code is entered into the mobile electronic device.

9. The method of claim 8, further comprising capturing a signature image using the mobile electronic device.

10. The method of claim 9, wherein the signature image is associated with an advanced care planning document of the patient.

11. The method of claim 4, wherein the located records are not part of the electronic medical record of the patient.

12. The method of claim 1, wherein the link is further preloaded with a private, shared key used to encrypt the URL parameter;
wherein the method further comprises validating the key responsive to the link being accessed; and
wherein decrypting the URL parameter comprises decrypting the URL parameter with the validated key.

13. The method of claim 1, further comprising accessing electronic medical record data of the patient stored in a cloud, wherein the electronic medical record data comprises data from one or more sources.

14. The method of claim 13, wherein the medical record data comprises an advance directive document, a power of attorney form, a living will, a do not resuscitate form, a physician orders for life sustaining treatment form, or a medical orders for life-sustaining treatment form.

15. The method of claim 1, wherein the URL parameter is encrypted before the link is preloaded with the URL parameter, and wherein the link is preloaded with the encrypted URL parameter.
